(19) United States
(12) Patent Application Publication
(10) Pub. No.: US 2005/0053601 A1
(43) Pub. Date: Mar. 10, 2005

(54) Novel g protein-coupled receptor protein and dna thereof

(76) Inventors: Takeo Moriya, Mino-shi, Osaka (JP); Takashi Ito, Tsukuba-shi Ibaraki (JP); Yasushi Shintani, Toyonaka-shi Osaka (JP); Hideki Matsui, Tsukuba-shi Ibaraki (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(21) Appl. No.: 10/258,362

(22) PCT Filed: Apr. 26, 2001
(86) PCT No.: PCT/JP01/03596

(30) Foreign Application Priority Data
Apr. 28, 2000 (JP) 2000-130470
May 12, 2000 (JP) 2000-140411

(51) Int. Cl. A61K 39/395; C07H 21/04; C07K 14/705; C07K 16/28

(52) U.S. Cl. 424/143.1; 435/69.1; 435/320.1; 435/325; 530/350; 530/388.22; 536/23.5

(57) ABSTRACT

A novel G protein-conjugating receptor protein containing an amino acid sequence which is the same or substantially the same as the amino acid sequence represented by SEQ ID NO.1, or its salt; a polynucleotide encoding the same; and medicinal use, etc. thereof.

### FIELD OF THE INVENTION

The present invention relates to a human brain-derived novel G protein-conjugating receptor protein or a salt thereof and a DNA encoding the same.

### BACKGROUND OF THE INVENTION

Many bioactive substances such as hormones and neurotransmitters serve to regulate the biological functions via specific receptor proteins which are present in cell membranes. Since most of such receptor proteins carry out an intracellular signal transmission via the activation of a guanine nucleotide-binding protein (hereinafter sometimes referred to as G protein) conjugating therewith and have a common structure containing 7 transmembrane regions, they are referred to together as G protein-conjugating receptor proteins or 7 transmembrane receptor proteins (7TMR).

The G protein-conjugating receptor protein is present on each functional cell surface in an in vivo cell or organ, and plays a physiologically important role as a target of a molecule which regulates the function of such cells or organs, for example, hormones, neurotransmitters and bioactive substances. A receptor transmits a signal to the inside of a cell via a binding to the bioactive substance, and such signal induces various reactions such as an activation or an inhibition of the cell.

Clarification of the relationship between a substance which regulates a complicated function in a range of in vivo cells or organs and the specific receptor protein, especially the G protein-conjugating receptor protein, serves to provide a valuable means for understanding the function in a range of in vivo cells or organs and for developing a pharmaceutical related closely to such function.

For example, the physiological function of in a range of in vivo organs is regulated under the control of a diversity of hormones, hormone-like substances, neurotransmitters and bioactive substances. Especially, bioactive substances are present in various in vivo locations, and regulates the physiological functions via the relevant receptor proteins. Many in vivo hormones, neurotransmitters and other bioactive substances still remain undiscovered, and their receptor protein structures have scarcely been reported. Moreover, even a known receptor protein had poorly be clarified whether its subtype exists or not.

Clarification of the relationship between a substance regulating a complicated in vivo function and a specific receptor protein is very important means for developing a pharmaceutical. In addition, for the purpose of screening efficiently an agonist and an antagonist for the receptor protein and developing a pharmaceutical, it is essential to understand the function of a gene of a receptor protein expressed in vivo and to express such protein in a suitable expression system.

Recently, a study on a random analysis of a cDNA sequence as a means for analyzing a gene expressed in vivo is performed intensively, and a fragment sequence of such cDNA thus obtained is registered and disclosed as Expressed Sequence Tags (ESTs) in a data base. However, most of such ESTs contain only sequence data, from which the relevant function is hardly assumed.

A substance inhibiting the binding between the G protein-conjugating receptor and the bioactive substance (i.e., ligand) or a substance causing a signal transmission similarly to a bioactive substance (i.e., ligand) as a result of a binding has been utilized as a specific antagonist or an agonist to such receptor in a pharmaceutical composition which regulates an in vivo function. Accordingly, to identify a novel G protein-conjugating receptor protein which is not only important in an in vivo physiological expression but also can serve as a target in developing a pharmaceutical and to clone its gene (for example, cDNA) serves as a very important means for identifying a novel G protein-conjugating receptor protein of specific ligand, agonist and antagonist.

Nevertheless, all G protein-conjugating receptors have not been identified, and there are currently still many undiscovered G protein-conjugating receptors and so-called orphan receptors whose relevant ligands are not identified, thus causing a demand for exploring a novel G protein-conjugating receptor as well as for clarifying the function thereof.

A G protein-conjugating receptor is useful for exploring a novel bioactive substance (i.e., ligand) and also for exploring an agonist or an antagonist for such receptor utilizing its signal transmitting effect as an index. On the other hand, an agonist or an antagonist for such receptor can be produced by analyzing the physiological effect of the receptor based on an inactivation experiment of the receptor (a knockout animal), even if no physiological ligand can be identified. A ligand, an agonist or an antagonist for such receptor is expected to be utilized as a prophylactic/therapeutic agent or a diagnostic agent for a disease related to the dysfunction of a G protein-conjugating receptor.

In addition, a reduced or increased in vivo function of a G protein-conjugating receptor resulted from a gene variation of such receptor frequently induces a certain disease. In such a case, a gene therapy by introducing the receptor gene into a living body (or into a certain organ) or by introducing an antisense nucleic acid into the receptor gene is possible in addition to the administration of an antagonist or an agonist to the receptor. For this purpose, the nucleotide sequence of the receptor is essential information for detecting a deletion or a variation in the gene, and a gene of the receptor is applicable to a prophylactic/therapeutic agent or a diagnostic agent for a disease related to the dysfunction of the receptor.

### SUMMARY OF THE INVENTION

The present invention is intended to provide a novel G protein-conjugating receptor protein which is useful as discussed above. Thus, the novel G protein-conjugating receptor protein or a partial peptide or a salt thereof, a polynucleotide (DNA, RNA or a derivative thereof) comprising a polynucleotide (DNA, RNA or a derivative thereof) encoding the G protein-conjugating receptor protein or the partial peptide thereof, a recombinant vector comprising the polynucleotide, a transformant containing the recombinant vector, a method for producing the G protein-conjugating receptor protein or a salt thereof, an antibody against the G protein-conjugating receptor protein or the partial peptide or a salt thereof, a compound which alters the expression level of the G protein-conjugating receptor protein, a method for determining a ligand for the G protein-conjugating receptor protein, a screening method for a compound (antagonist, agonist) or a salt thereof capable of altering the binding affinity between a ligand and the G protein-conjugating receptor protein, a kit for the screening, a compound (antagonist, agonist) or a salt there of capable of altering the binding affinity between a ligand and the G protein-conjugating receptor protein which can be obtained by using the screening method or the screening kit, and, a pharmaceutical composition comprising a compound (antagonist, agonist) capable of altering the binding affinity between a ligand and the G protein-conjugating receptor or a compound capable of altering the expression level of such G protein-conjugating receptor protein or a salt thereof are provided here.

The present inventors made an effort and was successful in isolating a cDNA encoding a novel G protein-conjugating receptor protein derived from a human brain and sequencing its entire bases. Then we found the first to seventh transmembrane regions on a hydrophobic plot based on the translation from the nucleotide sequence into an amino acid sequence, and ensured that the proteins encoded by these cDNAs were the G protein-conjugating receptor proteins of the 7 transmembrane region type. Based on these findings, the present inventors made a further effort and finally establish the invention.

Thus, the present invention relates to:
(1) a G protein-conjugating receptor protein comprising an amino acid sequence identical or substantially identical to the amino acid sequence represented by SEQ ID NO.1 or a salt thereof;
(2) a partial peptide of the G protein-conjugating receptor protein according to the above-mentioned (1) or a salt thereof;
(3) a polynucleotide comprising a polynucleotide encoding the G protein-conjugating receptor protein according to the above-mentioned (1);
(4) a polynucleotide according to the above-mentioned (3) which is a DNA;
(5) a polynucleotide according to the above-mentioned (3) having the nucleotide sequence represented by SEQ ID NO.2;
(6) a recombinant vector comprising the polynucleotide according to the above-mentioned (3);
(7) a transformant transformed with the recombinant vector according to the above-mentioned (6);
(8) a method for producing the G protein-conjugating receptor protein according to the above-mentioned (1) or a salt thereof, comprising cultivating the transformant according to the above-mentioned (7) to generate the G protein-conjugating receptor protein according to the above-mentioned (1);
(9) an antibody against the G protein-conjugating receptor protein according to the above-mentioned (1) or the partial peptide according to the above-mentioned (2) or a salt thereof;
(10) an antibody according to the above-mentioned (9) which is a neutralizing antibody which inactivates the signal transmission of the G protein-conjugating receptor protein according to the above-mentioned (1);
(11) a diagnostic composition comprising the antibody according to the above-mentioned (9);
(12) a ligand for the G protein-conjugating receptor protein according to the above-mentioned (1) or a salt thereof obtainable by using the G protein-conjugating receptor protein according to the above-mentioned (1) or the partial peptide according to the above-mentioned (2) or a salt thereof;
(13) a pharmaceutical composition comprising a ligand for the G protein-conjugating receptor protein according to the above-mentioned (12);
(14) a method for determining a ligand for the G protein-conjugating receptor protein according to the above-mentioned (1) or a salt thereof, comprising using the G protein-conjugating receptor protein according to the above-mentioned (1) or the partial peptide according to the above-mentioned (2) or a salt thereof;
(15) a method for screening a compound or a salt thereof which is capable of altering the binding affinity between a ligand and the G protein-conjugating receptor protein according to the above-mentioned (1) or a salt thereof, comprising using the G protein-conjugating receptor protein according to the above-mentioned (1) or the partial peptide according to the above-mentioned (2) or a salt thereof;
(16) a screening kit for a compound or a salt thereof capable of altering the binding affinity between a ligand and the G protein-conjugating receptor protein according to the above-mentioned (1) or a salt thereof, comprising the G protein-conjugating receptor protein according to the above-mentioned (1) or the partial peptide according to the above-mentioned (2) or a salt thereof;
(17) a compound or a salt thereof capable of altering the binding affinity between a ligand and the G protein-conjugating receptor protein according to the above-mentioned (1) or a salt thereof, which is obtainable by using the screening method according to the above-mentioned (15) or a screening kit according to the above-mentioned (16);
(18) a pharmaceutical composition comprising a compound or a salt thereof capable of altering the binding affinity between a ligand and the G protein-conjugating receptor protein according to the above-mentioned (1) or a salt thereof, which is obtainable by using the screening method according to the above-mentioned (15) or the screening kit according to the above-mentioned (16);
(19) a polynucleotide hybridizing with the polynucleotide according to the above-mentioned (3) under a high stringent condition;
(20) a polynucleotide comprising a nucleotide sequence or a part thereof which is complementary with the polynucleotide according to the above-mentioned (3);
(21) a method for quantifying an mRNA of the G protein-conjugating receptor protein according to the above-mentioned (1), comprising using the polynucloeotide according to the above-mentioned (3) or a part thereof;
(22) a method for quantifying the G protein-conjugating receptor protein according to the above-mentioned (1), comprising using the antibody according to the above-mentioned (9);
(23) a method for diagnosing a disease related to a function of the G protein-conjugating receptor protein according to the above-mentioned (1), comprising using the quantification method according to the above-mentioned (21) or (22);
(24) a screening method for a compound or a salt thereof capable of altering the expression level of the G protein-conjugating receptor protein according to the above-mentioned (1), comprising using the quantification method according to the above-mentioned (21);
(25) a screening method for a compound or a salt thereof capable of altering the amount of the G protein-conjugating receptor protein according to the above-mentioned (1) in a cell membrane, comprising using the quantification method according to the above-mentioned (22);
(26) a compound or a salt thereof capable of altering the expression level of the G protein-conjugating receptor protein according to the above-mentioned (1), which is obtainable by using the screening method according to the above-mentioned (24);
(27) a compound or a salt thereof capable of altering the amount of the G protein-conjugating receptor protein according to the above-mentioned (1) in a cell membrane, which is obtainable by using the screening method according to the above-mentioned (25);
(28) a pharmaceutical composition comprising a compound or a salt thereof capable of altering the expression level of the G protein-conjugating receptor protein according to the above-mentioned (1), which is obtainable by using the screening method according to the above-mentioned (24);
(29) a pharmaceutical composition comprising a compound or a salt thereof capable of altering the amount of the G protein-conjugating receptor protein according to the above-mentioned (1) in a cell membrane, which is obtainable by using the screening method according to the above-mentioned (25);
(30) the pharmaceutical composition according to the above-mentioned (18), (28) or (29) which is prophylactic or therapeutic against a disease in central nervous system, inflammatory disease, circulatory disease, cancer, diabetes or infertility;
(31) a prophylactic or therapeutic method for a disease in central nervous system, inflammatory disease, circulatory disease, cancer, diabetes or infertility, comprising administering to a mammal an effective amount of a compound or a salt thereof capable of altering the binding affinity between a ligand and the G protein-conjugating receptor protein according to the above-mentioned (1) or a salt thereof, which is obtainable by using the screening method according to the above-mentioned (15) or the screening kit according to the above-mentioned (16);
(32) a prophylactic or therapeutic method for a disease in central nervous system, inflammatory disease, circulatory disease, cancer, diabetes or infertility, comprising administering to a mammal an effective amount of a compound or a salt thereof capable of altering the expression level of the G protein-conjugating receptor protein according to the above-mentioned (1), which is obtainable by using the screening method according to the above-mentioned (24);
(33) a prophylactic or therapeutic method for a disease in central nervous system, inflammatory disease, circulatory disease, cancer, diabetes or infertility, comprising administering to a mammal an effective amount of a compound or a salt thereof capable of altering the amount of the G protein-conjugating receptor protein according to the above-mentioned (1) in a cell membrane, which is obtainable by using the screening method according to the above-mentioned (25);
(34) a use of a compound or a salt thereof capable of altering the binding affinity between a ligand and the G protein-conjugating receptor protein according to the above-mentioned (1) or a salt thereof, which is obtainable by using the screening method according to the above-mentioned (15) or the screening kit according to the above-mentioned (16), for producing a prophylactic or therapeutic agent against a disease in central nervous system, inflammatory disease, circulatory disease, cancer, diabetes or infertility;
(35) a use of a compound or a salt thereof capable of altering the expression level of the G protein-conjugating receptor protein according to the above-mentioned (1), which is obtainable by using the screening method according to the above-mentioned (24), for producing a prophylactic or therapeutic agent against a disease in central nervous system, inflammatory disease, circulatory disease, cancer, diabetes or infertility;
(36) a use of a compound or a salt thereof capable of altering the amount of the G protein-conjugating receptor protein according to the above-mentioned (1) in a cell membrane, which is obtainable by using the screening method according to the above-mentioned (25), for producing a prophylactic or therapeutic agent against a disease in central nervous system, inflammatory disease, circulatory disease, cancer, diabetes or infertility.

Those also provided are:
(37) the G protein-conjugating receptor protein according to the above-mentioned (1) or a salt thereof comprising [1] the amino acid sequence represented by Sequence ID. NO.1, an amino acid sequence formed as a result of the deletion of one or more (preferably approximately 1 to 30, more preferably approximately 1 to 9, most preferably several thereof (1 to 5)) amino acids in the amino acid sequence represented by Sequence ID. NO.1, [2] an amino acid sequence formed as a result of the addition of one or more (preferably approximately 1 to 30, more preferably approximately 1 to 10, most preferably several thereof (1 to 5)) amino acids to the amino acid sequence represented by Sequence ID. NO.1, [3] an amino acid sequence formed as a result of the substitution of one or more (preferably approximately 1 to 30, more preferably approximately 1 to 10, most preferably several thereof (1 to 5)) amino acids in the amino acid sequence represented by Sequence ID. NO.1 by other amino acids or [4] an amino acid sequence as a result of combination of [l]-[4];
(38) the method for determining a ligand according to the above-mentioned (14), comprising bringing the G protein-conjugating receptor protein according to the above-mentioned (1) or a salt thereof or the partial peptide according to the above-mentioned (2) or a salt thereof into contact with a test substance;
(39) the method for determining a ligand according to the above-mentioned (38) wherein said ligand is, for example, angiotensin, bombesin, cannabinoid, cholecystokinin, glutamin, serotonin, melatonin, neuropeptide Y, opioid, purine, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leucotriene, pancreastatin, prostaglandine, thromboxane, adenosine, adrenaline, α and β-chemokine (for example, IL-8, GROα, GROβ, GROγ, NAP-2, ENA-78, PF4, IP10, GCP-2, MCP-1, HC14, MCP-3, I-309, MIP1α, MIP-1β, RANTES and the like), endoserine, enterogastrin, histamine, neurotensin, TRH, pancreatic polypeptide, galanin, lyzophosphatidic acid (LPA) or sphingosine 1-phosphate;
(40) the screening method according to the above-mentioned (15) comprising comparing between (i) the case of a contact of the G protein-conjugating receptor protein according to the above-mentioned (1) or a salt thereof or the partial peptide according to the above-mentioned (2) or a salt thereof with a ligand, and (ii) the case of a contact of the G protein-conjugating receptor protein according to the above-mentioned (1) or the salt thereof or the partial peptide according to the above-mentioned (2) or the salt thereof with the ligand and a test compound;
(41) a screening method for a compound or a salt thereof capable of altering the binding affinity between a ligand and the G protein-conjugating receptor protein according to the above-mentioned (1) or a salt thereof, comprising determining and comparing between the levels of the binding of a labeled ligand to the G protein-conjugating receptor protein according to the above-mentioned (1) or the salt thereof or the partial peptide according to the above-mentioned (2) or a salt thereof upon (i) a contact of the labeled ligand with the G protein-conjugating receptor protein according to the above-mentioned (1) or the salt thereof or the partial peptide according to the above-mentioned (2) or the salt thereof, and upon (ii) a contact of the labeled ligand and a test compound with the G protein-conjugating receptor protein according to the above-mentioned (1) or the salt thereof or the partial peptide according to the above-mentioned (2) or the salt thereof;
(42) a screening method for a compound or a salt thereof capable of altering the binding affinity between a ligand and the G protein-conjugating receptor protein according to the above-mentioned (1) or a salt thereof, comprising determining and comparing between the levels of the binding to the cell of a labeled ligand upon (i) a contact of the labeled ligand with the cell comprising the G protein-conjugating receptor protein according to the above-mentioned (1), and upon (ii) a contact of the labeled ligand and a test substance with the cell comprising the G protein-conjugating receptor protein according to the above-mentioned (1);
(43) a screening method for a compound or a salt thereof capable of altering the binding affinity between a ligand and the G protein-conjugating receptor protein according to the above-mentioned (1) or a salt thereof, comprising determining and comparing between the levels of the binding of a labeled ligand to a membrane fraction of the cell upon (i) a contact of the labeled ligand with the membrane fraction of the cell comprising the G protein-conjugating receptor protein according to the above-mentioned (1), and upon (ii) a contact of the labeled ligand and a test substance with the membrane fraction of the cell comprising a G protein-conjugating receptor protein according to the above-mentioned (1);
(44) a screening method for a compound or a salt thereof capable of altering the binding affinity between a ligand and a G protein-conjugating receptor protein according to the above-mentioned (1) or a salt thereof expressed on the cell membrane of a transformant according to the above-mentioned (7) by cultivating said transformant, comprising determining and comparing between the levels of the binding of a labeled ligand to said G protein-conjugating receptor protein upon (i) a contact of the labeled ligand with said G protein-conjugating receptor protein, and upon (ii) a contact of the labeled ligand and a test substance with said G protein-conjugating receptor protein;
(45) a screening method for a compound or a salt thereof capable of altering the binding affinity between a ligand and the G protein-conjugating receptor protein according to the above-mentioned (1) or a salt thereof, comprising determining and comparing between the cell stimulating activities via the G protein-conjugating receptor protein upon (i) a contact of a compound activating the G protein-conjugating receptor protein according to the above-mentioned (1) or a salt thereof with a cell comprising the G protein-conjugating receptor protein according to the above-mentioned (1), and upon (ii) a contact of a compound activating the G protein-conjugating receptor protein according to the above-mentioned (1) or a salt thereof and a test compound with a cell comprising the G protein-conjugating receptor protein according to the above-mentioned (1);
(46) a method for screening a compound or a salt thereof capable of altering the binding affinity between a ligand and the G protein-conjugating receptor protein according to the above-mentioned (1) or a salt thereof, comprising determining and comparing between the cell stimulating activities via the G protein-conjugating receptor protein upon a contact of a compound activating the G protein-conjugating receptor protein according to the above-mentioned (1) or a salt thereof with the G protein-conjugating receptor protein expressed on the cell membrane of a transformant according to the above-mentioned (7) by cultivating said transformant, and upon a contact of a compound activating the G protein-conjugating receptor protein according to the above-mentioned (1) or a salt thereof and a test compound with the G protein-conjugating receptor protein expressed on the cell membrane of the transformant according to the above-mentioned (7) by cultivating said transformant;
(47) the screening method according to the above-mentioned (45) or (46) wherein the compound activating the G protein-conjugating receptor protein according to the above-mentioned (1) is angiotensin, bombesin, cannabinoid, cholecystokinin, glutamin, serotonin, melatonin, neuropeptide Y, opioid, purine, vasopressin, oxytocin, PACAP, secretin, glucagon, calcitonin, adrenomedulin, somatostatin, GHRH, CRF, ACTH, GRP, PTH, VIP (vasoactive intestinal polypeptide) somatostatin, dopamine, motilin, amylin, bradykinin, CGRP (calcitonin gene-related peptide), leucotriene, pancreastatin, prostaglandine, thromboxane, adenosine, adrenaline, α and β-chemokine (for example, IL-8, GROα, GROβ, GROγ, NAP-2, ENA-78, PF4, IP10, GCP-2, MCP-1, HC14, MCP-3, I-309, NIP1α, MIP-1β, RANTES and the like), endoserine, enterogastrin, histamine, neurotensin, TRH, pancreatic polypeptide, galanin, lyzophosphatidic acid (LPA) or sphingosine 1-phosphate;
(48) a compound or a salt thereof capable of altering the binding affinity between a ligand and the G protein-conjugating receptor protein according to the above-mentioned (1) or a salt thereof which is obtainable by the screening method according to the above-mentioned (40) to (47);
(49) a pharmaceutical composition comprising a compound or a salt thereof capable of altering the binding affinity between a ligand and the G protein-conjugating receptor protein according to the above-mentioned (1) or a salt thereof which is obtainable by the screening method according to the above-mentioned (40) to (47);
(50) the screening kit according to the above-mentioned (16) comprising a cell containing the G protein-conjugating receptor protein according to the above-mentioned (1);
(51) the screening kit according to the above-mentioned (16) comprising a membrane fraction of a cell including the G protein-conjugating receptor protein according to the above-mentioned (1);
(52) the screening kit according to the above-mentioned (16) comprising the G protein-conjugating receptor protein expressed on the cell membrane of a transformant according to the above-mentioned (7) by cultivating said transformant;
(53) a compound or a salt thereof capable of altering the binding affinity between a ligand and the G protein-conjugating receptor protein according to the above-mentioned (1) or a salt thereof, which is obtainable by using the screening kit according to the above-mentioned (50) to (52);
(54) a pharmaceutical composition comprising a compound or a salt thereof capable of altering the binding affinity between a ligand and the G protein-conjugating receptor protein according to the above-mentioned (1) or a salt thereof, which is obtainable by using the screening kit according to the above-memtioned (50) to (52);
(55) a method for quantifying the G protein-conjugating receptor protein according to the above-mentioned (1) or the partial peptide according to the above-mentioned (2) or a salt thereof, comprising bringing the antibody according to the above-mentioned (9) into contact with the G protein-conjugating receptor protein according to the above-mentioned (1) or the partial peptide according to the above-mentioned (2) or a salt thereof;
(56) a method for quantifying the G protein-conjugating receptor protein according to the above-mentioned (1) or the partial peptide according to the above-mentioned (2) or a salt thereof in a sample solution, comprising reacting competitively the antibody according to the above-mentioned (9) with the sample solution and a labelled G protein-conjugating receptor protein according to the above-mentioned (1) or partial peptide according to the above-mentioned (2) or a salt thereof, and determining the ratio of the labelled G protein-conjugating receptor protein according to the above-mentioned (1) or partial peptide according to the above-mentioned (2) or a salt thereof bound to the andibody; and,
(57) a method for quantifying the G protein-conjugating receptor protein according to the above-mentioned (1) or the partial peptide according to the above-mentioned (2) or a salt thereof in a sample solution, comprising reacting simultaneously or sequentially a sample solution with an antibody according to the above-mentioned (9) which is insolubilized on a support and a labelled antibody according to the above-mentioned (9), and determining the activity of the label on the insolubilized support.

### BRIEF DESCRIPTION OF THE DRAWINGS

FIG. 1 is a hydrophobicity plotting of TGR6.

FIG. 2 shows the amino acid sequence of SEQ ID NO.1 in a single-letter designation.

FIG. 3 show a graph representing tissue distribution of TGR6 expression.

### BEST MODE FOR CARRYING OUT THE INVENTION

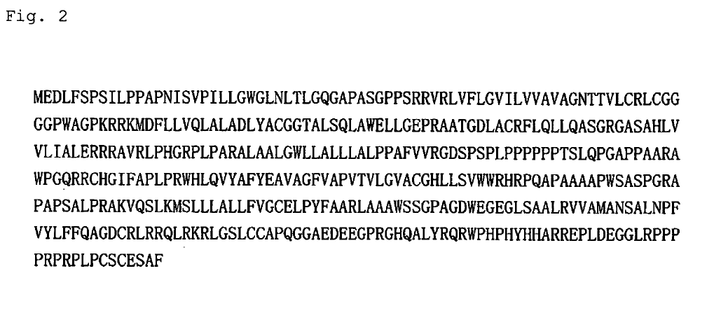

A G protein-conjugating receptor protein according to the invention (hereinafter sometimes referred to as a receptor protein) is a receptor protein comprising an amino acid sequence identical or substantially identical to the amino acid sequence represented by SEQ ID NO.1 (FIG. 2).

A receptor protein of the invention may be a protein derived for example from any of various cells (for example, splenic cell, neurocyte, gliacyte, pancreatic βcell, myelocyte, mesangial cell, Langerhans cell, epidermic cell, epithelial cell, endothelial cell, fibroblast, fibrocyte, myocyte, fat cell, immunocyte (e.g., macrophage, T cell, B cell, natural killer cell, mast cell, neutrophile, basophile, eosinophile, monocyte), megakaryocyte, synovial cell, chondrocyte, osteocyte, osteoblast, osteoclast, mammary gland cell, hepatocyte or interstitial cell, or a precursor cell therefor, stem cell or cancer cell and the like) or blood cells of a human or a mammalian animal (for example guinea-pig, rat, mouse, rabbit, swine, sheep, cattle, monkey and the like) or any tissue containing the cells listed above such as brain, brain parts (e.g., olfactory bulb, tonsillar nucleus, cerebral basal bulb, hippocampus, thalamus, hypothalamus, subthalamic nucleus, cerebral cortex, medulla oblongata, cerebellum, occipital lobe, frontal lobe, temporal lobe, putamen, caudatum, blush, nigra), spinal cord, pituitary gland, stomach, pancreas, kidney, liver, gonad, thyroid gland, gallbladder, bone marrow, adrenal gland, skin, muscle, lung, digestive tract (e.g., large intestine, small intestine), blood vessel, heart, thymus, spleen, submandibular gland, peripheral blood, peripheral blood cells, prostate, testis, orchis, ovary, placenta, uterus, bone, joint, skeletal muscle and the like as well as a synthetic protein.

An amino acid sequence substantially identical to the amino acid sequence represented by SEQ ID NO.1 may for example be an amino acid having about 50% or more, preferably about 60% or more, more preferably about 70% or more, further preferably about 80% or more, still further preferably about 90% or more, and most preferably about 95% or more of the homology with the amino acid sequence represented by SEQ ID NO.1.

Preferably, a protein comprising an amino acid sequence according to the invention which is substantially identical to the amino acid sequence represented by SEQ ID NO.1 may for example be a protein having an amino acid sequence substantially identical to the amino acid sequence represented by SEQ ID NO.1 and having an activity substantially similar to the amino acid sequence represented by SEQ ID NO.1.

An activity which is substantially similar may for example be a ligand binding activity and signal transmitting activity. The expression “substantially similar” means that the activity is similar qualitatively to each other. Accordingly, the ligand binding activity or the signal transmitting activity may be different in the degree of the activity or in a quantitative factor such as the molecular weight of the protein, while such activity is preferably comparable (e.g., about 0.01 to 100 times, preferably about 0.5 to 20 times, more preferably about 0.5 to 2 times).

While the determination of the activity such as the ligand binding activity and the signal transmitting activity can be performed in accordance with a method known per se, a ligand determination and a screening method described below may for example be employed.

A receptor protein according to the invention may also be a protein having [1] an amino acid sequence formed as a result of the deletion of one or more (preferably 1 to about 30, more preferably 1 to about 10, most preferably 1 to 5) amino acids in the amino acid sequence represented by Sequence ID. NO.1, [2] an amino acid sequence formed as a result of the addition of one or more (preferably 1 to about 30, more preferably 1 to about 10, most preferably 1 to 5) amino acids to the amino acid sequence represented by Sequence ID. NO.1, [3] an amino acid sequence formed as a result of the substitution of one or more (preferably 1 to about 30, more preferably 1 to about 10, most preferably 1 to 5) amino acids in the amino acid sequence represented by Sequence ID. NO.1 by other amino acids or [4] an amino acid as a result of combination of [1]-[4].

A receptor protein according to the invention has an N-terminal (amino terminal) in its left end a C-terminal (carboxyl terminal) in the right end as ordinarily in the peptide designation. While a receptor protein of the invention including a receptor protein comprising the amino acid sequence represented by SEQ ID NO.1 as its representative usually has a carboxyl group (—COOH) or a carboxylate (—COO⁻) at its C-terminal, it may have an amide (—CONH₂) or an ester (—COOR) at its C-terminal.

R in an ester employed here may for example be a C₁₋₆ alkyl group such as methyl, ethyl, n-propyl, isopropyl and n-butyl, and a C₃₋₈ cycloalkyl group such as cyclopentyl and cyclohexyl, and a C₆₋₂ aryl group such as phenyl, and α-naphthyl and a C₇₋₁₄ aralkyl including a phenyl-C₁₋₂ alkyl such as benzyl and phenethyl, or an α-naphthyl-C₁₋₂ alkyl such as α-naphthylmethyl, as well as a pivaloyloxymethyl group employed widely as an oral ester.

When a receptor protein of the invention has a carboxyl group (or carboxylate) anywhere other than its C-terminal, it may also be included in a receptor protein of the invention when such carboxyl group is amidated or esterified. The ester in such case may be an ester in the C-terminal listed above.

A receptor protein of the invention also includes one whose amino group in the methionine residue at the N-terminal of a protein described above is protected by a protective group (for example, C₁₋₆ acyl group including a C₂₋₆ alkanoyl group such as formyl and acetyl groups), one whose glutamyl group generated as a result of an in vivo cleavage of the N-terminal is converted into a pyroglutamic acid, one whose substituent on a side chain of an intramolecular amino acid (for example, —OH, —SH, amino group, imidazole group, indole group, guanidino group) is protected by a suitable protective group (for example, C₁₋₆ acyl group including a C₂₋₆ alkanoyl group such as formyl and acetyl groups), or a conjugated protein such as a glycoprotein having a sugar chain bound thereto.

Concretely, a receptor protein of the invention may for example be a receptor protein comprising the amino acid sequence represented by SEQ ID NO.1.

A partial peptide of a receptor protein of the invention (hereinafter sometimes abbreviated as a partial peptide) may be any partial peptide of a receptor protein of the invention described above, and may for example be a site of a receptor protein of the invention which is exposed to the outside of a cell membrane and which has a receptor binding activity.

Concretely, a partial peptide of the receptor protein having the amino acid sequence represented by SEQ ID NO.1 may for example be a peptide containing a part which is proven to be a extracellular region (hydrophilic site) in a hydrophobic plotting analysis. A peptide partially containing a hydrophobic site may also be employed similarly. While a peptide containing each domain discretely may also be employed, a peptide containing several domains simultaneously may also be employed.

The number of the amino acids in a partial peptide of the invention is preferably at least 20, more preferably at least 50 and most preferably at least 100, such amino acids being selected from the constituent amino acids of a receptor protein of the invention.

A substantially identical amino acid sequence means an amino acid sequence having about 50% or more, preferably about 60% or more, more preferably about 70% or more, further preferably about 80% or more, still further preferably about 90% or more, and most preferably about 95% or more of the homology with the relevant amino acid sequence.

The term “substantially similar activity” employed here has the meaning similar to that described above. A determination of such “substantially similar activity” can be performed as described above.

A partial peptide of the invention may be subjected to the deletion of one or more (preferably 1 to about 10, more preferably 1 to 5) amino acids in an amino acid sequence described above, the addition of one or more (preferably 1 to about 20, more preferably 1 to 10, most preferably 1 to 5) amino acids to the amino acid sequence and the substitution of one or more (preferably 1 to about 10, more preferably 1 to 5) amino acids in the amino acid sequence.

While a partial peptide of the invention usually has a carboxyl group (—COOH) or a carboxylate (—COO⁻) at its C-terminal, it may have an amide (—CONH₂) or an ester (—COOR) at its C-terminal similarly to a protein of the invention.

A partial peptide of the invention also includes, similarly to a receptor protein of the invention, one whose amino group in the methionine reside at the N-terminal is protected by a protective group, one whose Gln formed as a result of an in vivo cleavage at the N-terminal is converted into a pyroglutamic acid, one whose substituent on a side chain of an intramolecular amino acid is protected by a suitable protective group, or a conjugated protein such as a glycoprotein having a sugar chain bound thereto.

While a partial peptide of the invention usually has a carboxyl group (—COOH) or a carboxylate (—COO⁻) at its C-terminal, it may have an amide (—CONH₂) or an ester (—COOR) at its C-terminal similarly to a protein of the invention.

A salt of a receptor protein of the invention or a partial peptide thereof may for example be a physiologically acceptable salt with an acid or a base, and a physiologically acceptable acid addition salt is preferred especially. Such salt may for example be a salt with an inorganic acid (for example, hydrochloric acid, phosphoric acid, hydrobromic acid, sulfuric acid), or with an organic acid (for example, acetic acid, formic acid, propionic acid, fumaric acid, maleic acid, succinic acid, tartaric acid, citric acid, malic acid, oxalic acid, benzoic acid, methanesulfonic acid, benzenesulfonic acid).

A receptor protein of the invention or a salt there of may be produced by a known method for purifying a protein from a cell or a tissue of human or a mammalian animal described above, or may be produced by cultivating a transformant comprising a DNA encoding a receptor protein of the invention as described below. Alternatively, a protein synthesis described below or an analogous method may also be employed.

When a tissue or a cell of human or a mammalian animal is used as a starting material, it is homogenized and extracted, for example, with an acid to obtain an extract, which is then purified by a combination of chromatographic methods such as a reverse phase chromatography, an ion exchange chromatography and the like.

To synthesize a receptor protein or a partial peptide of the invention or its salt or amide, usually a commercial resin for protein synthesis may be used. Such resin may for example be a chloromethyl resin, a hydroxymethyl resin, a benzhydrylamine resin, an aminomethyl resin, a 4-benzyloxybenzyl alcohol resin, a 4-methylbenzhydrylamine resin, a PAM resin, a 4-hydroxymethylmethylphenylacetamide resin, a polyacrylamide resin, a 4-(2′,4′-dimethoxyphenyl-hydroxymethyl)phenoxy hydroxymethyl)phenoxy resin, a 4-(2′,4′-dimethoxyphenyl-Fmoc aminoethyl)phenoxy resin and the like. Using such resin, an amino acid whose a-amino group and side chain functionalities are protected suitably is condensed on the resin according to any condensation method known per se in the order of the sequence of an intended protein. At the end of the reaction, a protein is isolated from the resin with being deprotected simultaneously, and then subjected to an intramolecular disulfide bond forming reaction in a highly diluted solution to obtain an intended protein or its amide.

While the condensation of a protected amino acid described above may be effected using various activating reagents which can be employed for synthesizing a protein, a carbodiimide is employed preferably. Such carbodiimide may be DCC, N,N′-diisopropylcarbodiimide, N-ethyl-N′-(3-dimethylaminopropyl)carbodiimide and the like. For an activation using any of those listed above, a protected amino acid may be added directly to a resin together with a racemization-inhibiting auxiliary agent (for example, HOBt, HOOBt), or a protected amino acid may previously be activated as a symmetric acid anhydride or an HOBt ester or an HOOBt ester and then added to a resin.

A solvent used in an activation of a protected amino acid or in a condensation with a resin may be one selected from the solvents known to be useful in a protein condensation reaction. Those employed may for example be an acid amide such as N,N-dimethylformamide, N,N-dimethylacetamide and N-methylpyrrolidone, a halogenated hydrcarbon such as methylene chloride and chloroform, an alcohol such as trifluoroethanol, a sulfoxide such as dimethylsulfoxide, an ether such as pyridine, dioxane and tetrahydrofuran, a nitrile such as acetonitrile and propionitrile, an ester such as methyl acetate and ethyl acetate, or a mixture thereof. The reaction temperature may appropriately be selected from the range known to be useful in a protein binding reaction, and may usually range from −20° C. to 50° C. An activated amino acid derivative is employed usually in excess of 1.5 to 4 times. When a ninhydrin reaction test revealed an insufficient condensation, the condensation is repeated without any deprotection to achieve a sufficient condensation. When a repetitive condensation is still not successful in achieving a sufficient condensation, acetic anhydride or acetylimidazole may be employed for acetylating an unreacted amino acid.

A protective group for an amino group of a starting material may for example be Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulfenyl, diphenylphosphonothioyl, Fmoc and the like.

A carboxyl group can be protected for example by an alkylesterification (for example a straight, branched or cyclic alkylesterification employing methyl, ethyl, propyl, butyl, t-butyl, cyclopentyl, cyclohexyl, cycloheptyl, cyclooctyl, 2-adamantyl and the like), an aralkylesterification (for example benzylesterification, 4-nitrobenzylesterification. 4-methoxybenzylesterification, 4-chlorbenzylesterification, benzhydrylesterification), phenacylesterification and can also be protected as benzyloxycarbonylhydrazide, t-butoxycarbonylhydrazide, tritylhydrazide and the like.

The hydroxyl group of serine can be protected for example by an esterification or an etherification. A group suitable in such esterification may for example be a lower alkanoyl group such as an acetyl group, an aroyl group such as a benzoyl group, or a group derivatized from carbonic acid such as a benzyloxycarbonyl group and an ethoxycarbonyl group. A group suitable in such etherification may for example be a benzyl, tetrahydropyranyl and t-butyl groups.

A protective group for a phenolic hydroxyl group of tyrosine may for example be Bzl, C₂-Bzl, 2-nitrobenzyl, Br-Z, t-butyl and the like.

A protective group for imidazole of histidine may for example be Tos, 4-methoxy-2,3,6-trimethylbenzenesulfonyl, DNP, benzyloxymethyl, Bum, Boc, Trt, Fmoc and the like.

A starting substance whose carboxyl group is activated may for example be a corresponding acid anhydride, an azide, an activated ester [ester with alcohol (for example pentachlorophenol, 2,4,5-trichlorophenol, 2,4-dinitrophenol, cyanomethyl alcohol, p-nitrophenol, HONB, N-hydroxysuccinimide, N-hydroxyphthalimide, HOBt)]. A starting substance whose amino group is activated may for example be a corresponding amide phaophate.

A method for a deprotection (cleavage) may be a catalytic hydrogenation under a hydrogen flow in the presence of a catalyst such as Pd-black or Pd—C, a treatment with an acid such as anhydrous hydrofluoric acid, methanesulfonic acid, trifluoromethanesulfonic acid, trifluoroacetic acid or a mixture thereof, a treatment with a base such as diisopropylethylamine, triethylamine, piperidine, piperazine and the like, as well as a reduction with sodium in a liquid ammonia. A cleavage employing an acid treatment described above is performed usually at a temperature of —20° C. to 40° C., and such acid treatment is effected advantageously by adding a cation scavenger such as anisol, phenol, thioanisol, m-cresol, p-cresol, dimethylsulfide, 1,4-butanedithiol, 1,2-ethanedithiol and the like. A 2,4-dinitrophenyl group employed as an imidazole protective group for histidine is deprotected by a thiophenol treatment, while a formyl group employed as an indole protective group of tryptophan is removed by a deprotection using an acid treatment in the presence of 1,2-ethanedithiol and 1,4-butandithiol as described above and also by a treatment with an alkali such as a diluted solution of sodium hydroxide and a diluted ammonia.

A protection of a functional group which should not be involved in a reaction, a protective group therefor, a deprotection of such protective group and an activation of a functional group involved in a reaction may appropriately be selected from the groups and the methods known per se.

In an alternative method for obtaining an amide of a protein, the a-carboxyl group of the amino acid at the carboxy terminal may for example be protected as being amidated, and then a peptide chain (protein) is elongated to a desired length in the direction of its amino group, and then a protein from which only the protection group of the α-amino group at the N-terminal of the peptide and a protein from which only the protection group of the carboxyl group at the C-terminal of are produced, and the both proteins are condensed in a solvent mixture described above. Such condensation is detailed above. After purifying a protected protein obtained by the condensation, all protective groups are removed by the methods described above to yield a desired crude protein. This crude protein can be purified by any of the known purification means and a target fraction is lyophilized to obtain an amide of the desired protein.

To obtain an ester of a protein, the α-carboxyl group of the amino acid at the carboxy terminal may for example be condensed with a desired alcohol to form an amino acid ester, which is converted into an ester of a desired protein similarly to the case of an amide of a protein.

A partial peptide of a protein of the invention or its salt can be produced according to a peptide synthesis method known per se or by cleaving a protein of the invention with an appropriate peptidase. Such peptide synthesis method may be a solid phase synthesis or a liquid phase synthesis. Thus, a partial peptide or an amino acid capable of constituting a protein of the invention is condensed with the remainder moiety and then a protective group, if any, of the product is removed to obtain an intended peptide. Examples of the known condensation methods and deprotection methods are described in References [1] to [5] shown below.
[1] M. Bodanszky and M. A. Ondetti, Peptide Synthesis, Interscience Publishers, New York (1966);
[2] Schroeder and Luebke, The Peptide, Academic Press, New York (1965);
[3] N. Izumiya et al, Basics and experiments of peptide synthesis, Maruzen (1975);
[4] H. Yajima et al, Biochemisty experiment 1, Protein chemistry IV, 205 (1977);
[5] H. Yajima (supervision), Pharmaceutical development II, Vol.14, Peptide synthesis, Hirokawa Shoten.

After the reaction, a partial peptide of the invention can be isolated and purified by a combination if a solvent extraction, a distillation, a column chromatography, a liquid chromatography, a recrystallization and the like. When the partial peptide thus obtained is in a free form then it can be converted into a suitable salt by a known method, and when the product is a salt then it can be converted into a free form or other salts by a known method.

A polynucleotide encoding a receptor protein of the invention may be any polynucleotide comprising a nucleotide sequence which encodes the receptor protein of the invention described above (DNA or RNA, preferably DNA). Such polynucleotide may be a DNA or an RNA such as an mRNA which encodes a receptor protein of the invention, and which may be double-stranded or single-stranded. A double-stranded polynucleotide may be a double-stranded DNA, a double-stranded RNA or a hybrid of DNA:RNA. A single-stranded polynucleotide may be a sense (i.e., encoding) strand or an antisense (i.e., non-encoding) strand.

Using a polynucleotide encoding an receptor protein of the invention and in accordance with a known method for example that described in JIKKEN IGAKU-ZOKAN “, “New PCR and its application”, 15(7), 1997, an mRNA of an inventive receptor can be quantified.

A DNA encoding a receptor protein of the invention may be a genome DNA, a genome DNA library, a cDNA derived from a cell or a tissue described above, a cDNA library derived from a cell or a tissue described above and a synthetic DNA. A vector employed to obtain a library may be a bacteriophage, a plasmid, a cosmid, a phagimid and the like. A total RNA or an mRNA fraction prepared from a cell or a tissue described above may also be used directly in an amplification by a Reverse Transcriptase Polymerase Chain Reaction (hereinafter abbreviated as RT-PCR).

Concretely, a DNA encoding a receptor protein of the invention may for example be a DNA comprising the nucleotide sequence represented by SEQ ID NOs.2 or a DNA having a nucleotide sequence capable of being hybridized under a high stringent condition with the nucleotide sequence represented by SEQ ID NO.2 and encoding a receptor protein having an activity substantially similar to that of the protein of the invention (e.g., ligand binding activity, signal transmitting activity).

A DNA capable of being hybridized with the nucleotide sequence represented by SEQ ID NO.2 may for example be a DNA having about 70% or more, preferably about 80% or more, more preferably about 90% or more and most preferably about 95% or more of the homology with the nucleotide sequence represented by SEQ ID NO.2.

A hybridization can be performed by a method known per se or its modification, for example, a method described in Molecular Cloning, 2nd, J. Sambrook et al., Cold Spring Harbor Lab. Press (1989). When a commercial library is employed, an attached instruction may be followed. More preferably, a high stringent condition is employed.

Such a high stringent condition may for example be one employing a sodium concentration of about 19 to 40 mM, preferably about 19 to 20 mM and a temperature of about 50 to 70° C., preferably about 60 to 65° C. A sodium concentration of about 19 mM combined with a temperature of about 65° C. is most preferred.

More concretely, a DNA encoding a receptor protein having the amino acid sequence represented by SEQ ID NO.1 may be a DNA having the nucleotide sequence represented by SEQ ID NO.2.

A polynucleotide comprising a part of the nucleotide sequence of a DNA encoding a receptor protein of the invention or a part of the nucleotide sequence complementary with such DNA is intended not only to comprise the DNA encoding a partial peptide of the invention described above but also to comprise an RNA.

According to the invention, an antisense polynucleotide (nucleic acid) capable of inhibiting the replication or the expression of a G protein-conjugating receptor protein gene can be designed and synthesized based on the nucleotide sequence data of a DNA which is cloned or encodes a determined G protein-conjugating receptor protein. Such polynucleotide (nucleic acid) can be hybridized with an RNA of a G protein-conjugating receptor protein gene whereby inhibiting the synthesis or the function of such RNA, or can interact with a G protein-conjugating receptor protein-related RNA whereby regulating or controlling the expression of a G protein-conjugating receptor protein gene. A polynucleotide complementary with a selected sequence of a G protein-conjugating receptor protein-related RNA and a polynucleotide capable of being hybridized specifically with a G protein-conjugating receptor protein-related RNA are useful in regulating and/or controlling the in vivo and in vitro expression of a G protein-conjugating receptor protein gene and is useful also in treating or diagnosing a disease. The expression “corresponding to” is used herein to mean a homology to or a complementarity with a certain sequence of a nucleotide including a gene, a base or a nucleic acid. The expression “corresponding to” when used here in the context of the relationship of a nucleotide, a nucleotide sequence and a peptide (protein) usually means an amino acid of a peptide (protein) to be derived from the nucleotide (nucleic acid) sequence or a sequence complementary therewith. While a 5′-end hairpin loop, a 5′-end 6 base pair repeat, a 5′-end non-translation region, a polypeptide translation initiation codon, a protein-encoding region, an ORF translation initiation codon, a 3′-end non-translation region, a 3′-end palindrome region and a 3′-end hairpin loop of a G protein-conjugating receptor protein gene can be selected as a preferred target region, any region in the G protein-conjugating receptor protein gene can also be selected as a target region.

The relationship between an intended nucleic acid and a polynucleotide complementary with at least a part of a target region, i.e., the relationship with a polynucleotide capable of being hybridized with a target can be regarded to be “antisense”. An antisense polynucleotide may for example be a polydeoxynucleotide containing 2-deoxy-D-ribose, a polydeoxynucleotide containing D-ribose, a polynucleotide of any other type which is an N-glycoside of a purine or pyrimidine base, any other polymer containing a non-nucleotide backbone (for example, a commercially available protein nucleic acid and a synthetic sequence-specific nucleic acid polymer) or any other polymer containing a special binding (provided that such polymer contains a nucleotide having a configuration capable of accepting a base pairing or a base adhesion found in a DNA or an RNA). It may be a double-stranded DNA, a single-stranded DNA, a double-stranded RNA, a single-stranded RNA, a DNA:RNA hybrid, a unmodified polynucleotide (or a unmodified oligonucleotide), a polynucleotide having a known modification, for example, one having a known label, a capped polynucleotide, a methylated polynucleotide, a polynucleotide obtained by substituting one or more natural nucleotides with analogues, a polynucleotide whose intramolecular nucleotides are modified, for example, one having a non-charged bond (for example, methylphosphonate, phosphotriester, phosphoramidate, carbamate), one having a charged bond or a sulfur-containing bond (for example, phosphorothioate, phosphorodithioate), for example, one having a side chain residue such as a protein (nuclease, nuclease inhibitor, toxin, antibody, signal peptide, poly-L-lysine) or a sugar (for example, monosaccharide), one having an intercalating compound (for example, acridine, psoralen), one containing a chelating compound (for example, metal, radioactive metal, boron, oxidative metal), one containing an alkylating agent, one having a modified bond (for example, α-anomer nucleic acid) and the like. The terms “nucleoside”, “nucleotide” and “nucleic acid” employed here may include a form containing not only purine and pyrimidine bases but also other modified heterocyclic bases. Such modified form may contain methylated purine and pyrimidine, acylated purine and pyrimidine as well as other heterocyclic rings. A modified nucleoside and a modified nucleotide may be further modified at their sugar moiety, in which, for example, one or more hydroxyl groups are substituted by halogens or aliphatic groups, or converted into a functional group such as an ether or an amine.

An antisense polynucleotide (nucleic acid) of the invention is an RNA, a DNA, or a modified nucleic acid (RNA, DNA). Concretely, such modified nucleic acid includes, but not limited to a sulfur derivative or a thiophosphate derivative of a nucleic acid, and those which are resistant to a polynucleosideamide or oligonucleosideamide degradation. An antisense nucleic acid of the invention can preferably be designed based on the policy described below. Thus, an effort is made for the purpose of achieving a higher intracellular stability of an antisense nucleic acid, a higher cellular permeability of an antisense nucleic acid, a higher affinity with a target sense strand, and a lower toxicity, if any, of an antisense nucleic acid.

Such modifications are reported extensively in this field, as disclosed for example in J. Kawakami et al., Pharm Tech Japan, Vol.8, pp.247, 1992; Vol.8, pp.395, 1992; S. T. Crooke et al. ed., Antisense Research and Applications, CRC Press, 1993.

An antisense nucleic acid of the invention can be altered or may contain a modified sugar, base or bond, and can be provided in a special form such as a liposome or a microsphere, applied to an gene therapy or can be given as an adduct. Such adduct may be a polycation such as a polylysine which serves to neutralize the electric charge of a phosphate backbone, or a hydrophobic material such as a lipid (for example, phospholipid, cholesterol) which serves to enhance the interaction with a cell membrane and to promote the intake of a nucleic acid. A lipid to be added preferably is cholesterol or a derivative thereof (for example, cholesteryl chloroformate, cholic acid) and the like. These are capable of being attached to the 3′-or 5′-end of a nucleic acid, can be attached via a base, a sugar or an intracellular nucleoside bond. Other groups may for example be a group for a capping located specifically at the 3′- or 5′-end of a nucleic acid, such as those intended to avoid the degradation by a nuclease such as exonucleases or RNases. A group for such capping includes, but not limited to a protective group for a hydroxyl group known in the art such as a glycol including polyethylene glycol and tetraethylene glycol, which are not limiting.

The inhibitory activity of an antisense nucleic acid can be examined using a transformant of the invention, an in vivo or in vitro gene expression system of the invention or a in vivo or in vitro translation system of a G protein-conjugating receptor protein. Such nucleic acid can be applied to a cell by a method known per se.

A DNA encoding a partial peptide of the invention may be any DNA containing a nucleotide sequence encoding a partial peptide of the invention described above. It may be a genome DNA, a genome DNA library, a cDNA derived from a cell or a tissue described above, a cDNA library derived from a cell or a tissue described above and a synthetic DNA. A vector employed to obtain a library may be a bacteriophage, a plasmid, a cosmid, a phagimid and the like. An mRNA fraction prepared from a cell or a tissue described above may also be used directly in an amplification by a Reverse Transcriptase Polymerase Chain Reaction (hereinafter abbreviated as RT-PCR).

Concretely, a DNA encoding a partial peptide of the invention may for example be [1] a DNA having a partial nucleotide sequence of a DNA comprising the nucleotide sequence represented by SEQ ID NO.2 or [2] a DNA having a partial nucleotide sequence of a DNA having a nucleotide sequence capable of being hybridized under a high stringent condition with the nucleotide sequence represented by SEQ ID NO.2 and encoding a receptor protein having an activity substantially similar to that of the protein of the invention (e.g., ligand binding activity, signal transmitting activity).

A DNA capable of being hybridized with the nucleotide sequence represented by SEQ ID NO.2 may for example be a DNA having about 70% or more, preferably about 80% or more, more preferably about 90% or more and most preferably about 95% or more of the homology with the nucleotide sequence represented by SEQ ID NO.2.

In a method for cloning a DNA which completely encodes a receptor protein or a partial peptide thereof according to the invention (hereinafter sometimes abbreviated as a receptor of the invention) a synthetic DNA primer having a partial nucleotide sequence of the receptor protein of the invention is employed in an amplification by a PCR, or a selection is made by means of a hybridization of a DNA integrated into a suitable vector and labeled with a DNA fragment encoding a part or all of the receptor protein of the invention or with a synthetic DNA. A hybridization can be performed for example by a method described in Molecular Cloning, 2nd, J. Sambrook et al., Cold Spring Harbor Lab. Press (1989). When a commercial library is employed, an attached instruction may be followed.

The conversion of the nucleotide sequence of a DNA can be performed by a PCR or a known kit, such as Mutan™-super Express KmG (Takara) or Mutan™-K (Takara), in accordance with a method known per se or a modification thereof, such as ODA-LA PCR method, Gupped duplex method or Kunkel method.

A cloned receptor protein-encoding DNA can be used directly or after a digestion with a restriction enzyme or an addition of a linker if desired. Such DNA may have an ATG as a translation initiation codon at its 5′ terminal and a TAA, TGA or TAG as a translation termination codon at its 3′ terminal. Such translation initiation or termination codon may be added using an appropriate synthetic DNA adapter.

An expression vector for a receptor protein of the invention can be prepared for example by (i) cutting a desired DNA fragment out of a DNA encoding the receptor protein of the invention, and (ii) ligating said DNA fragment to the downstream of a promoter of a suitable expression vector.

Such a vector may for example be an E.coli-derived plasmid (e.g., pBR322, pBR325, pUC12, pUC13), a B.subtilis-derived plasmid (e.g., pUB110, pTP5, pC194), an yeast-derived plasmid (e.g., pSH19, pSH15), a bacteriophage such as λ phage, an animal virus such as retrovirus, vaccinia virus, vaculovirus and the like, as well as pA1-11, pXT1, pRc/CMV, pRC/RSV, pcDNAI/Neo and the like.

A promoter employed in the invention may be any promoter which is appropriate correspondingly to a host employed for expressing a gene. For example, when an animal cell is employed as a host cell, those exemplified are SRαpromoter, SV40 promoter, LTR promoter, CMV promoter, HSV-TK promoter and the like.

Among those listed above, CMV promoter and SRα promoter are preferably employed. Those preferred for an Escherichia as a host cell are trp promoter, lac promoter, recA promoter, λPL promoter, lpp promoter and the like, those preferred for a Bacillus as a host cell are SPO1 promoter, SPO2 promoter, penP promoter and the like, those preferred for an yeast as a host cell are PHO5 promoter, PGK promoter, GAP promoter, ADH promoter and the like. Those preferred for an insect cell as a host cell are polyhedrin promoter, P10 promoter and the like.

In addition to those described above, an expression vector containing, if desired, an enhancer, a splicing signal, a polyA addition signal, a selection marker, an SV 40 replication origin (hereinafter sometimes abbreviated as SV40ori) and the like may also be employed. A selection marker may for example be a dihydrofolic acid reductase (hereinafter sometimes abbreviated as dhfr) gene [methotrexate (MTX) resistant], an ampicillin resistant gene (hereinafter sometimes abbreviated as Amp^{r}), a neomycin resistant gene (hereinafter sometimes abbreviated as Neo^{r}, G418 resistant) and the like. Especially when a dhfr gene is employed as a selection marker using a CHO (dhfr−) cell, an intended gene can be selected using a thymidine-free medium.

A signal sequence suitable for a host cell may also be added if necessary to the N-terminal of a receptor protein of the invention. Those preferred for an Escherichia as a host cell are Pho A signal sequence, Omp A signal sequence and the like, those preferred for a Bacillus as a host cell are α-amylase signal sequence, subtilicin signal sequence and the like, those preferred for an yeast as a host cell are MFα signal sequence, SUS2 signal sequence and the like, and those preferred for an animal cell as a host cell are insulin signal sequence, α-interferon signal sequence, an antibody molecule signal sequence and the like.

Using a vector comprising a DNA encoding a receptor protein of the invention thus constructed, a transformant can be prepared.

A host cell may for example be an Escherichia, a Bacillus, an yeast, an insect cell, an insect, an animal cell and the like.

Such Escherichia may for example be Escherichia coli K12 DH1 (Proc. Natl. Acad. Sci. USA, Vol.60, 160 (1968)), JM103 (Nucleic Acids Research, Vol.9, 309 (1981)), JA221 (Journal of Molecular Biology, Vol.120, 517 (1978)), HB101 (Journal of Molecular Biology, Vol.41, 459 (1969)), C600 (Genetics, Vol.39, 440 (1954)) and the like.

A Bacillus may for example be Bacillus subtilis MI114 (Gene, Vol.24, 255 (1983)), 207-21 (Journal of Biochemistry, Vol.95, 87 (1984)) and the like.

An yeast may for example be Saccharomyces cerevisiae AH22, AH22R⁻, NA87-11A, DKD-5D, 20B-12, Schizosaccharomyces pombe NCYC1913, NCYC2036, Pichia pastoris KM71 and the like.

An insect cell, when a virus is AcNPV, may for example be an armyworm-derived cultured cell line, (Spodoptera frugiperda cell; Sf cell), a Trichoplusia ni mesenteron-derived MG1 cell, Trchoplusia ni egg-derived High Five TM cell, Mamestra brassicae-derived cell, Estigmena acrea-derived cell and the like. A cell when a virus is BmNPV may for example be a silkworm-derived cultured cell line (Bombyx mori N cell; BmN cell) and the lie. Such Sf cell may for example be an Sf9 cell (ATCC CRL1711), an Sf21 cell (for both, see Vaughn, J. L. et al., In Vivo, 13, 213-217 (1977)) and the like.

An insect may for example be a larva of a silkworm (Maeda et al., Nature, Vol.315, 592 (1985)).

An animal cell may for example be a simian cell COS-7, Vero, a Chinese hamster cell CHO (hereinafter abbreviated as CHO cell), a dhfr gene-defect Chinese hamster cell CHO (hereinafter abbreviated as CHO(dhfr⁻) cell), a mouse L cell, a mouse AtT-20, a mouse myeloma cell, a rat GH3, a human FL cell and the like.

An Escherichia can be transformed for example by a method described in Proc. Natl. Acad. Sci. USA, Vol.69, 2110 (1972), or Gene, Vol.17, 107 (1982).

A Bacillus can be transformed for example by a method described in Molecular and General Genetics, Vol.168, 111 (1979).

An yeast can be transformed for example by a method described in Methods in Enzymology, Vol.194, 182-187 (1991), Proc. Natl. Acad. Sci. USA, Vol.75, 1929 (1978) and the like.

An insect cell or an insect can be transformed for example by a method described in Bio/Technology, 6, 47-55 (1988).

An animal cell can be transformed for example by a method described in CELL ENGINEERING EXTRA ISSUE No.8, NEW CELL ENGINEERING EXPERIMENTAL PROTOCOL, 263-267 (1995) (SHUJUNSHA), Virology, Vl.52, 456 (1973) and the like.

As described above, a transformant which had been transformed with an expression vector comprising a DNA encoding a G protein-conjugating receptor protein can be obtained.

When cultivating a transformant whose host cell is an Escherichia or a Bacillus, a suitable culture medium employed is a liquid medium which may contain substances required for the growth of the relevant transformant such as carbon sources, nitrogen sources, inorganic substances and the like. A carbon source may for example be glucose, dextrin, soluble starch, sucrose and the like, and a nitrogen source may for example be an inorganic or organic material such as an ammonium salt, a nitrate, corn steep liquor, peptone, casein, meat extract, soybean bran, potato extract and the like, and an inorganic substance may for example be calcium chloride, sodium dihydrogen phosphate, magnesium chloride and the like. An yeast extract, a vitamin and a growth promoting factor may also be added. The pH of a medium is preferably about 5 to 8.

A preferred culture medium for cultivating an Escherichia may for example a M9 medium containing glucose and casamino acid (Miller, Journal of Experiments in Molecular Genetics, 431-433, Cold Spring Harbor Laboratory, New York (1972)) . If necessary, a medium may contain an agent for facilitating the action of a promoter such as 3β-indolyl acrylic acid.

When an Escherichia is employed as a host cell, the cultivation is performed usually at 15 to 43° C. for about 3 to 24 hours, if necessary with an aeration or a stirring.

When a Bacillus is employed as a host cell, the cultivation is performed usually at 30 to 40° C. for about 6 to 24 hours, if necessary with an aeration or a stirring.

When cultivating a transformant whose host cell is an yeast, a suitable culture medium employed may for example be a Burkholder minimum medium (Bostian, K. L. et al., Proc. Natl. Acad. Sci. USA, Vol.77, 4505 (1980), and a 0.5% casamino acid-supplemented SD medium (Bitter, G. A. et al., Proc. Natl. Acad. Sci. USA, Vol.81, 5330 (1984). The pH of a culture medium is adjusted preferably at about 5 to 8. The cultivation is performed usually at 20 to 35° C. for about 24 to 72 hours, if necessary with an aeration or a stirring.

When cultivating a transformant whose host cell is an insect cell or an insect, a suitable culture medium employed may for example be a Grace's Insect Medium (Grace, T. C. C., Nature, 195, 788 (1962) supplemented appropriately, for example, with an inactivated 10% bovine serum. The pH of a culture medium is adjusted preferably at about 6.2 to 6.4. The cultivation is performed usually at 27° C. for about 3 to 5 days, if necessary with an aeration or a stirring.

When cultivating a transformant whose host cell is an animal cell, a suitable culture medium employed may for example be a MEM medium supplemented with about 5 to 20% fetal bovine serum (Science, Vol.122, 501 (1952), a DMEM medium (Virology, Vol.8, 396 (1959), an RPMI1640 medium (The Journal of the American Medical Association, Vol.199, 519 (1967), a 199 medium (Proceedings of the Society for the Biological Medicine, Vol.73, 1 (1950), and the like. The pH of a culture medium is preferably about 6 to 8. The cultivation is performed usually at 30 to 40° C. for about 15 to 60 hours, if necessary with an aeration or a stirring.

As described above, a G protein-conjugating receptor protein according to the invention can be produced in an intracellular region, in a cell membrane or in an extracellular region of a transformant.

In order to separate and purify a receptor protein of the invention from a cell culture described above, a method described below may for example be employed.

For extracting a receptor protein of the invention from a cultured microorganism or a cultured cell, the cell is collected by a known method after an cultivation and suspended in a suitable buffer solution, which is subjected to an ultrasonication, a treatment with lysozyme and/or a freezing and thawing cycle to destruct the cell followed by a centrifugation or a filtration to obtain a crude extract of the receptor protein. The buffer solution may contain a protein denaturing agent such as urea or guanidine hydrochloride or a surfactant such as Triton X-100™. When a receptor protein is secreted into a culture medium, then a cell and a supernatant are separated by a method known per se after completing the cultivation to collect the supernatant.

The purification of a receptor protein contained in a culture supernatant or an extract thus obtained may be performed by an appropriate combination of separation and purification methods known per se. Such known separation and purification methods are a method utilizing a solubility such as a salting out or a solvent precipitation, a method mainly utilizing the difference in the molecular weight such as a dialysis, a ultrafiltration, a gel filtration and an SDS-polyacrylamide gel electrophoresis, a method utilizing the difference in the electric charge such as an ion exchange chromatography, a method utilizing the difference in the hydrophobicity such as a reverse phase high pressure liquid chromatography, a method utilizing the difference in the isoelectric point such as an isoelectric focusing and the like.

When a receptor protein thus obtained is in a free form then it can be converted into a salt by a method known per se or its modification, and, on the contrary, when it is obtained as a salt then it can be converted into a free form or another salt by a method known per se or its modification.

It is also possible that a protein produced by a recombinant is treated with a suitable protein-modifying enzyme before or after a purification to achieve a desired modification or a partial removal of a polypeptide. Such protein-modifying enzyme may for example be trypsin, chymotrypsin, arginyl endopeptidase, protein kinase, glycosidase and the like.

A receptor protein of the invention or its salt thus produced can be examined for its activity for example by a labelled ligand binding test or an enzyme immunoassay employing a specific antibody.

An antibody against a receptor protein or a partial peptide of the invention or its salt may be either a polyclonal antibody or a monoclonal antibody provided that it can recognize such receptor protein or partial peptide of the invention or its salt.

An antibody against a receptor protein or a partial peptide of the invention or its salt (hereinafter abbreviated as a receptor protein of the invention) can be produced using the receptor protein of the invention as an antigen by a known method for producing an antibody or antiserum.

[Monoclonal Antibody Preparation]

(a) Preparation of Monoclonal Antibody-Producing Cell

A receptor protein of the invention can be administered as it is or in combination with a carrier or diluent to a site where the antibody can be produced in response to the administration to a mammalian animal. The administration may be combined with an administration of a Freund's complete adjuvant or a Freund's incomplete adjuvant for the purpose of enhancing the antibody-producing ability. The administration is performed usually once per 2 to 6 weeks, 2 to 10 times in total. A mammalian animal employed may for example be monkey, rabbit, dog, guinea-pig, mouse, rat, sheep and goat, with mouse and rat being employed preferably.

For preparing a monoclonal antibody-producing cell, antigen-immunized warm-blooded animals, for example mice, are screened for an individual exhibiting an antibody titre, from which a spleen or a lymph node is extracted 2 to 5 days after the final immunization, and an antibody-producing cell contained therein is fused with a myeloma cell, whereby preparing a monoclonal antibody-producing hybridoma. The antibody titre of an antiserum can be determined for example by reacting a labelled protein described below with the antiserum followed by determining the activity of the label bound to an antibody. The fusion can be accomplished by a known method such as one by Kohler and Milstein (Nature, 256, 495 (1975)). A fusion promoting agent may for example be a polyethylene glycol (PEG) or Sendai virus, with PEG being employed preferably.

A myeloma cell may for example be NS-1, P3U1, SP2/0, with P3U1 being employed preferably. A preferred ratio of the antibody-producing cell count (spleen cell count) and the myeloma cell count employed is about 1:1 to 20:1, and an efficient cell fusion is accomplished by adding a PEG (preferably PEG 1000 to PEG 6000) at a concentration of 10 to 80% and cultivating at 20 to 40° C., preferably 30 to 37° C., for 1 to 10 minutes.

While various methods are applicable in screening for a monoclonal antibody-producing hybridoma, those which may be exemplified are a method involving an addition of a hybridoma culture supernatant to a solid phase (e.g., a microplate) on which a antigen such as a receptor protein is adsorbed directly or in combination with a carrier followed by an addition of an anti-immunoglobulin labelled with a radioactive substance or with an enzyme (anti-mouse immunoglobulin antibody is employed when the cell employed in the cell fusion is a mouse cell) or protein A whereby detecting a monoclonal antibody bound to the solid phase, or a method involving an addition of a hybridoma culture supernatant to a solid phase on which an anti-immunoglobulin antibody or protein A followed by an addition of a receptor protein labelled with a radioactive substance or with an enzyme whereby detecting a monoclonal antibody bound to the solid phase.

While a monoclonal antibody can be selected by a method known per se or its modification, a HAT (hypoxanthine, aminopterin, thymidine)-supplemented medium for an animal cell culture is employed usually. A medium for the selection and the breeding may be any medium capable of growing a hybridoma. For example, an RPMI 1640 medium supplemented with 1 to 20%, preferably 10 to 20% fetal bovine serum, a GIT medium (Wako Pure Chemical) supplemented with 1 to 10% fetal bovine serum and a serum-free medium for cultivating a hybridoma (SFM-101, NISSUI SEIYAKU) may be employed. The cultivation temperature is usually 20 to 40° C., preferably about 37° C. The cultivation time is usually 5 days to 3 weeks, preferably 1 week to 2 weeks. The cultivation may be performed usually under an atmosphere of 5% CO₂ gas. The antibody titre of a hybridoma cultivation supernatant can be determined similarly to an antibody titre of an antiserum described above.

(b) Purification of Monoclonal Antibody

A monoclonal antibody can be separated and purified, similarly to a standard method for separating and purifying a polyclonal antibody, such as a method for separating and purifying an immunoglobulin [e.g., salting out, alcohol precipitation, isoelectric precipitation, electrophoresis, ion exchanger (e.g., DEAE) adsorption and desorption, ultracentrifugation, gel filtration, a specific purification for collecting an antibody exclusively using an antigen-binding solid phase or an active adsorbent such as protein A or protein G followed by dissociating the binding to obtain the antibody].

[Polyclonal Antibody Preparation]

A polyclonal antibody of the invention can be prepared by a method known per se or its modification. For example, a complex of an immune antigen (antigen such as a receptor protein) with a carrier protein is produced and used as described above in the section of the polyclonal antibody preparation to immunize a mammalian animal, from which a material containing an antibody against a receptor protein of the invention is isolated and purified to obtain an antibody.

With regard to a complex of an immune antigen with a carrier protein employed for immunizing a mammalian animal, the type of the carrier protein and the mixing ratio of the carrier and a hapten may vary provided that the antibody can be produced efficiently in relation to the hapten crosslinked to the carrier for the immunization, and any substance can be crosslinked at any ratio, and, in a typical method, about 0.1 to 20, preferably about 1 to 5 of parts by weight of bovine serum albumin, bovine thyroglobulin or keyhole limpet hemocyanin is coupled to 1 parts by weight of a hapten.

For coupling a hapten to a carrier, various condensing agent can be employed, such as gurtaraldehyde or carbodiimide, a maleimide activated ester, an active ester reagent having a thiol group or a dithiopyridyl group.

A condensation product may can be administered as it is or in combination with a carrier or diluent to a site of a warm-blooded animal where the antibody can be produced. The administration may be combined with an administration of a Freund's complete adjuvant or a Freund's incomplete adjuvant for the purpose of enhancing the antibody-producing ability. The administration is performed usually once per 2 to 6 weeks, 3 to 10 times in total.

A polyclonal antibody can be collected from a blood, ascites, preferably from a blood, of a warm-blooded animal immunized as described above.

The polyclonal antibody titre of an antiserum can be determined similarly to the measurement of the antibody titre of an antiserum described above. A polyclonal antibody can be isolated and purified in accordance with a method for isolating and purifying an immunoglobulin similar to a method for isolating and purifying a monoclonal antibody described above.

A receptor protein of the invention or a salt thereof, a partial peptide there of or a salt thereof and a DNA encoding such receptor protein or a partial peptide thereof can be used in (1) a determination of a ligand (agonist) for a G protein-conjugating receptor protein of the invention, (2) a prophylactic and/or therapeutic composition against a disease related to the dysfunction of a G protein-conjugating receptor protein of the invention, (3) a gene diagnostic agent, (4) a method for screening a compound capable of altering the expression level of a receptor protein of the invention or a partial peptide thereof, (5) a prophylactic and/or therapeutic composition against various diseases containing a compound capable of altering the expression level of a receptor protein of the invention or a partial peptide thereof, (6) a method for quantifying a ligand for a G protein-conjugating receptor protein of the invention, (7) a method for screening a compound (agonist, antagonist) capable of altering the binding affinity between a G protein-conjugating receptor protein of the invention and a ligand, (8) a prophylactic and/or therapeutic composition against various diseases containing a compound (agonist, antagonist) capable of altering the binding affinity between a G protein-conjugating receptor protein of the invention and a ligand, (9) a quantification of a receptor protein of the invention or a partial peptide thereof or a salt thereof, (10) a method for screening a compound capable of altering the amount of a receptor protein of the invention or a partial peptide thereof in a cell membrane, (11) a prophylactic and/or therapeutic composition against various diseases containing a compound capable of altering the amount of a receptor protein of the invention or a partial peptide thereof in a cell membrane, (12) a neutralization of a receptor protein of the invention or a partial peptide thereof or a salt thereof by an antibody, and (13) a creation of a non-human animal having a DNA encoding a G protein-conjugating receptor protein of the invention.

Especially by using a receptor binding assay system employing an inventive recombinant G protein-conjugating receptor protein expression system, a compound (e.g., agonist and antagonist) altering the binding affinity of a ligand to a human- or mammal-specific G protein-conjugating receptor can be screened and such agonist or antagonist can be employed in a prophylactic or therapeutic composition against any relevant disease.

The uses of a receptor protein of the invention or a partial peptide or a salt thereof (hereinafter sometimes abbreviated as a receptor protein and the like of the invention), a DNA encoding a receptor protein of the invention or a partial peptide or a salt thereof (hereinafter sometimes abbreviated as a DNA of the invention) and an antibody directed to a receptor protein and the like of the invention are discussed in detail below.

(1) Determination of Ligand (Agonist) for G Protein-Conjugating Receptor Protein of the Invention

A receptor protein of the invention or a salt there of or a partial peptide of the invention or a salt thereof is useful as a reagent for searching for or determining a ligand (agonist) for a receptor protein of the invention or a salt thereof.

Thus, the present invention provides a method for determining a ligand for a receptor protein of the invention comprising bringing a receptor protein of the invention or a salt there of or a partial peptide of the invention or a salt thereof into contact with a test compound.

Such test compound may for example be a known ligand (for example, angiotensin, bombesin, cannabinoid, cholecystokinin, glutamin, serotonin, melatonin, neuropeptide Y, opioid, purine, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leucotriene, pancreastatin, prostaglandine, thromboxane, adenosine, adrenaline, α and β-chemokine (for example, IL-8, GROα, GROβ, GROγ, NAP-2, ENA-78, PF4, IP10, GCP-2, MCP-1, HC14, MCP-3, I-309, NIP1α, MIP-1β, RANTES and the like), endoserine, enterogastrin, histamine, neurotensin, TRH, pancreatic polypeptide or galanin, lyzophosphatidic acid (LPA) or sphingosine 1-phosphate) as well as an extract of a tissue of a human or an mammalian animal (for example, mouse, rat, swine, cattle, sheep and monkey) and a cell culture supernatant. For example, such tissue extract of cell culture supernatant may be added to a receptor protein of the invention and fractionated while determining a cell stimulating activity and the like, whereby obtaining a single ligand finally.

Concretely, in a ligand determination method of the invention, a receptor protein or a partial peptide thereof is employed, or a recombinant receptor protein expression system is constructed and used in a receptor binding assay system, whereby determining a compound (for example, peptide, protein, non-peptide compound, synthetic compound, fermentation product and the like) or a salt thereof having a cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) as being bound to the receptor protein of the invention.

A ligand determination method of the invention is characterized by a determination of the binding of a test substance to a receptor protein of the invention or a partial peptide thereof or the cell stimulating activity when bringing such receptor protein of the invention or the partial peptide thereof into contact with such test compound.

More concretely, the present invention provides:
[1] a method for determining a ligand for a receptor protein of the invention or a salt thereof, comprising determining the level of the binding of a labeled test compound to the receptor protein of the invention or the salt there of or a partial peptide of the invention or a salt thereof when bringing such labeled test compound into contact with such protein or the salt thereof or such partial peptide or the salt thereof;
[2] a method for determining a ligand for a receptor protein of the invention or a salt thereof, comprising determining the level of the binding of a labeled test compound to a cell containing the receptor protein of the invention or a membrane fraction of such cell when bringing such labeled test compound into contact with such cell or such membrane fraction;
[3] a method for determining a ligand for a receptor protein of the invention, comprising determining the level of the binding of a labeled test compound to the receptor protein expressed on a cell membrane by cultivating a transformant containing a DNA encoding the receptor protein of the invention when bringing such labeled test compound into contact with such receptor protein or a salt thereof;
[4] a method for determining a ligand for a receptor protein of the invention or a salt thereof, comprising determining a receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) when bringing a labeled test compound into contact with a cell containing the receptor protein of the invention; and,
[5] a method for determining a ligand for a receptor protein of the invention or a salt thereof, comprising determining a receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) when bringing a labeled test compound into contact with a receptor protein expressed on a cell membrane by cultivating a transformant containing a DNA encoding the receptor protein of the invention.

It is preferred particularly to perform the tests [1] to [3] described above to ensure the binding of a test compound to a receptor protein of the invention prior to performing the tests [4] to [5] described above.

While a receptor protein employed primarily in a method for determining a ligand may be any of receptor proteins of the invention or those containing a partial peptide of the invention described above, a receptor protein expressed in a large amount using an animal cell is suitable.

In order to produce a receptor protein of the invention, an expression method described above may be employed, and it is preferred to express a DNA encoding such receptor protein in a mammalian cell or an insect cell. While a complementary DNA is employed usually as a DNA fragment encoding a target protein moiety, it is not essential. For example, a gene fragment or a synthetic DNA may also be employed. For the purpose of introducing a DNA fragment encoding a receptor protein of the invention into an animal host cell to effect a highly efficient expression, it is preferred to integrate such DNA fragment into the downstream of a polyhedron promoter of a nuclear polyhedrosis virus (NPV) classified as a vaculovirus whose host is an insect, an SV 40-derived promoter, a retrovirus promoter, a metallothioneine promoter, a human heat shock promoter, a cytomegalovirus promoter, an SRα promoter and the like. An expressed receptor can be examined for its quantity or quality by a method known per se. For example, a method known in a literature (Nambi, P. et al., J.Biol.Chem., Vo.267, p.19555 to 19559, 1992) may be employed.

Accordingly, in a method for determining a ligand of the invention, those containing a receptor protein of the invention or a partial peptide or a salt thereof may be a receptor protein or a partial peptide or a salt thereof which was purified by a method known per se or a cell containing such a receptor protein or a cell membrane fraction obtained therefrom.

When a cell containing a receptor protein of the invention is employed in a method for determining a ligand of the invention, this cell may be immobilized by glutaraldehyde or formalin. Such immobilization can be effected by a method known per se.

A cell containing a receptor protein of the invention is a host cell expressing the receptor protein of the invention, and such host cell may be an Escherichia, a Bacillus, an yeast, an insect cell, an animal cell and the like.

A cell membrane fraction means a cell membrane-rich fraction obtained by a method known per se after pelletizing the cell. A cell may be pelletized for example by a method in which a cell is pressed and crashed by a Potter-Elvehjem homogenizer, by using a whirling blender or a polytron (Kinematica), by means of an ultrasonic treatment, or by a method in which a cell is sprayed via a fine nozzle while being pressurized by a French press. A cell membrane may be fractionated mainly by a centrifugal fractionation such as a fractional centrifugation or a density gradient centrifugation. For example, a cell pellet is centrifuged at a low speed (500 rpm to 3000 rpm) for a short period (usually about 1 minute to 10 minutes) to obtain a supernatant, which is then centrifuged at a higher speed (15000 rpm to 30000 rpm) usually for 30 minutes to 2 hours to obtain a pellet, which is used as a membrane fraction. This membrane fraction contains a large amount of the membrane components such as an expressed receptor protein and phospholipids and membrane proteins derived from the cell.

The amount of a receptor protein in a cell containing such receptor protein or a membrane fraction thereof is preferably 10³ to 10⁸ molecules, more preferably 10⁵ to 10⁷ molecules per cell. A higher expression leads to a higher ligand binding activity (specific activity) per membrane fraction, whereby allowing not only a highly sensitive screening system to be established but also a large amount of a sample to be determined in an identical lot.

For performing the methods [1] to [3] described above for determining a ligand for a receptor protein of the invention or a salt thereof, a suitable receptor protein fraction and a labeled test compound are required.

A receptor protein fraction is preferably a natural receptor protein fraction or a recombinant receptor fraction having an activity which is equivalent to that of the natural one. The expression “activity which is equivalent” employed here means an equivalent ligand binding activity or signal transmission activity.

A labeled test compound may for example be [³H]-, [¹²⁵I]-, [¹⁴C]- or [³⁵S]-labeled angiotensin, bombesin, cannabinoid, cholecystokinin, glutamin, serotonin, melatonin, neuropeptide Y, opioid, purine, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leucotriene, pancreastatin, prostaglandine, thromboxane, adenosine, adrenaline, α and β-chemokine (for example, IL-8, GROα, GROβ, GROγ, NAP-2, ENA-78, PF4, IP10, GCP-2, MCP-1, HC14, MCP-3, I-309, MIP1α, MIP-1β, RANTES and the like), endoserine, enterogastrin, histamine, neurotensin, TRH, pancreatic polypeptide, galanin lyzophosphatidic acid (LPA) and sphingosine 1-phosphate.

Concretely for performing a method for determining a ligand for a receptor protein of the invention or a salt thereof, a cell containing the receptor protein of the invention or a membrane fraction of the cell is suspended in a buffer suitable for the determination method to prepare a receptor standard. Such buffer may for example be a buffer which is not inhibit the binding between a ligand and the receptor protein, such as a phsophate buffer or tris-HCl buffer, pH 4 to 10 (preferably pH 6 to 8). Also for the purpose of reducing the non-specific binding, a surfactant such as CHAPS, Tween-80™ (KAO-ATLAS), digitonin, deoxycholate and the like or various protein such as bovine serum albumin and gelatin may be added to a buffer. In addition, for the purpose of suppressing the cleavage of a receptor or a ligand by a protease, a protease inhibitor such as PMSF, leupeptine, E-64 (PEPTIDE INSTITUTE, INC) and a pepstatin may also be added. In a 0.01 ml to 10 ml of a solution of the receptor described above, a certain amount (5000 cpm to 500000 cpm) of [³H]—, [125I]—, [¹⁴C]— or [³⁵S]-labeled test substance is contained. For measuring the non-specific binding (NSB) level, a reaction tube containing a large excess of the non-labeled test compound may also be provided. The reaction is performed at a temperature of about 0° C. to 50° C., preferably about 4° C. to 37° C. for a period of about 20 minutes to 24 hours, preferably about 30 minutes to 3 hours. After the reaction, the mixture is filtered through a filter such as a glass fiber filter paper and washed with an appropriate volume of the same buffer and then the radioactivity remaining on the glass fiber filter paper is determined by a liquid scintillation counter or a gamma-counter. A test compound whose count (B—NSB) obtained by subtracting the non-specific binding (NSB) level from the total binding (B) level exceeded 0 cpm can be selected as a ligand (agonist) for the receptor protein of the invention or a salt thereof.

In order to perform the methods [4] to [5] described above for determining a ligand for a receptor protein of the invention or a salt thereof, such receptor protein-mediated cell stimulating activity (for example, an activity which promotes or suppresses arachidonic acid release, acetylcholin release, intracellular Ca²+ release, intracellular cAMP production, intracellular cGMP production, inositol phosphate production, cell membrane potential variation, intracellular protein phosphorylation, c-fos activation, pH reduction and the like) can be determined by a known method or using a commercial assay kit. Concretely, a cell containing a receptor protein is cultivated in a multi-well plate. For determining a ligand, the medium is replaced with a fresh buffer or an appropriate buffer which is non-toxic to the cell, and a test compound is added and incubated for a certain period, and then the cell is extracted or the supernatant is recovered and the product accumulated is quantified by a relevant method. When the production of a substance which is an index for a cell stimulating activity (for example, arachidonic acid) is difficult to be assayed due to any degrading enzyme contained in the cell, an inhibitor of such degrading enzyme may be added to perform the assay. Another activity such as a cAMP production inhibiting activity can be detected as a production inhibiting effect in a cell whose basal production has been increased using forskolin and the like.

A kit of the invention for determining a ligand which binds to a receptor protein of the invention or a salt thereof comprises the receptor protein of the invention or a salt thereof, a partial peptide of the invention or a salt thereof, a cell containing the receptor protein of the invention or a membrane fraction of a cell containing the receptor protein of the invention.

A kit of the invention for determining a ligand may for example be one of those listed below.

1. Reagents for Ligand Determination

[1] Assay Buffer and Washing Buffer

Hanks' Balanced Salt Solution (Gibco) supplemented with 0.05% bovine serum albumin (Sigma).

The buffer is sterilized by filtering through a filter whose pore size is 0.45 μm, and may be stored at 4° C. or prepared just before use.

[2] Preparation of G Protein-Conjugating Receptor Protein

A CHO cell expressing a receptor protein of the invention is subjected to a subculture in a 12-well plate at the density of 5×10⁵ cells/well and cultivated at 37° C. under 5% CO₂ and 95% air for 2 days.

[3] Labeled Test Compound

A commercially available [³H]—, [125I]—, [¹⁴C]— or [³⁵S]-labeled compound or an appropriately labeled compound.

A compound in an aqueous solution is stored at 4° C. or −20° C. and diluted at 1 μM with an assay buffer just before use. A water-insoluble test compound is dissolved in dimethylformamide, DMSO, methanol and the like.

[4] Non-Labeled Test Compound

The compound identical to the labeled compound is prepared at a concentration higher by 100 to 1000 times.

2. Assay Procedures

[1] CHO cell expressing the receptor protein of the invention, which has been cultivated in a 12-well tissue culture plate, is washed twice with 1 ml of an assay buffer and each 490 μl of the assay buffer is added to each well.

[2] 5 μl of a labeled test compound was added and allowed to react at room temperature for 1 hour. 5 μl of a non-labeled test compound is also added for measuring the non-specific binding.

[3] The reaction mixture is removed and the cell is washed three times with 1 ml of the washing buffer. The labeled test compound bound to the cell is dissolved by 0.2 N NaOH— 1% SDS, and mixed with 4 ml of a liquid scintillator A (Wako Pure Chemical).

[4] Using a liquid scintillation counter (Beckman), the radioactivity is determined.

A ligand capable of being bound to a receptor protein of the invention or a salt thereof may for example be a substance existing specifically in brain, pituitary and pancreas, such as angiotensin, bombesin, cannabinoid, cholecystokinin, glutamin, serotonin, melatonin, neuropeptide Y, opioid, purine, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leucotriene, pancreastatin, prostaglandine, thromboxane, adenosine, adrenaline, α and β-chemokine (for example, IL-8, GROα, GROβ, GROγ, NAP-2, ENA-78, PF4, IP10, GCP-2, MCP-1, HC14, MCP-3, I-309, MIP1α, MIP-1β, RANTES and the like), endoserine, enterogastrin, histamine, neurotensin, TRH, pancreatic polypeptide, galanin, lyzophosphatidic acid (LPA) or sphingosine 1-phosphate, and the like.

(2) Prophylactic and/or Therapeutic Agent Against Disease Related to Dysfunction of G Protein-conjugating Receptor Protein of the Invention

Once a ligand for a receptor protein of the invention was characterized in the method (1) described above, it becomes possible to use [1] the receptor of the invention or [2] a DNA encoding this receptor protein as a pharmaceutical for preventing and/or treating a disease related to the dysfunction of the receptor protein of the invention.

For example, in a patient whose ligand is not allowed to exert a physiological effect due to an in vivo reduction in a receptor protein of the invention (a patient having a deficiency of the receptor), the in vivo receptor protein in this patient can be increased to allow the ligand to exert the effect sufficiently by [1] administering the receptor protein of the invention to the patient to supplement the receptor protein, or by [2] (a) administering a DNA encoding the receptor protein of the invention to the patient to effect an expression of the protein or (b) inserting a DNA encoding the receptor protein of the invention into a target cell where the protein is expressed followed by implanting the cell into the patient. Thus, a DNA encoding a receptor protein of the invention is useful as a safe and poorly toxic pharmaceutical for preventing and/or treating a disease related to the dysfunction of the receptor protein of the invention.

A receptor protein of the invention is a novel 7 transmembrane receptor protein having a homology of about 28% and about 27%, at the level of the amino acid sequence, with a murine oxytocin receptor and human oxytocin receptor, respectively, which are one of G protein-conjugating receptor proteins.

A receptor protein of the invention is useful in preventing and/or treating a disease in central nervous system (for example, Alzheimer's disease, dementia, eating disorder), inflammatory disease (for example, allergy, asthma, rheumatism), circulatory disease (for example, hypertension, hypercardia, angina pectris, arterial sclerosis), cancer (for example, non-small cell lung cancer, ovarian cancer, prostatic cancer, gastric cancer, bladder cancer, mammary cancer, uterine cervix cancer, colon cancer, rectal cancer), diabetes, infertility and the like.

When a receptor protein of the invention is used as a prophylactic and/or therapeutic agent as discussed above, a standard method can be employed to obtain a dosage form.

On the other hand, when a DNA encoding a receptor protein of the invention (hereinafter sometimes abbreviated as a DNA of the invention) is used as a prophylactic and/or therapeutic agent described above, the DNA of the invention can be administered directly as it is or after an insertion into a suitable vector such as a retrovirus vector, an adenovirus vector, an adenovirus-associated virus vector and the like by a standard method. A DNA of the invention can be administered, as it is or in a formulation together with an auxiliary agent for promoting an ingestion, using a gene gun or a catheter such as a hydrogel catheter.

For example, [1] a receptor protein of the invention or [2] a DNA encoding such receptor protein may be given orally as an optionally sugar-coated tablet, capsule, elixir, microcapsule and the like, or parenterally as a formulation for injection such as an aseptic solution or suspension in water or pharmacological acceptable liquid. For example, such formulation can be produced by mixing [1] a receptor protein of the invention or [2] a DNA encoding such receptor protein with a known physiologically acceptable carrier, flavor, excipient, vehicle, preservative stabilizer, binder and the like in a unit dosage form which is acceptable generally in a pharmaceutical practice. The amount of an active ingredient in such formulation should be adjusted to achieve a suitable dose within a specified range.

An additive which may be incorporated into a tablet or a capsule may for example be a binder such as gelatin, corn starch, tragacanth, gum arabic and the like, an excipient such as crystalline cellulose, an expander such as corn starch, gelatin, alginic acid and the like, a lubricant such as magnesium stearate, a sweetener such as sucrose, lactose or saccharin, a flavor such as peppermint, oil of Geultheria ovatifolia spp., cherry and the like. When a unit dosage form is a capsule, a liquid carrier such as a fat may further be incorporated in addition to the materials described above. An aseptic formulation for injection can be prepared in accordance with an ordinary pharmaceutical practice such as a dissolution or a suspension of an active ingredient, a naturally-occurring vegetable oil such as sesame oil and palm oil in a vehicle such as a water for injection. An aqueous liquid for injection may for example be physiological saline, an isotonic solution containing glucose or other auxiliary agents (for example, D-sorbitol, D-mannitol, sodium chloride) and the like, which may be used in combination with an suitable solubilizer such as an alcohol (for example, ethanol), a polyalcohol (for example, propylene glycol, polyethylene glycol), a non-ionic surfactant (for example, polysorbate 80™, HCO-50). An oily liquid may for example be sesame oil and soybean oil, which may be used in combination with an solubilizer such as benzyl benzoate, benzyl alcohol and the like.

In addition, a prophylactic and/or therapeutic agent described above may be supplemented also with a buffer agent (for example, phosphate buffer, sodium acetate buffer), an analgesic (for example, benzalkonium chloride, procaine hydrochloride), a stabilizer (for example, human serum albumin, polyethylene glycol), a preservative (for example, benzyl alcohol, phenol), an antioxidant. A formulation for injection thus prepared is then filled usually in a suitable ampoule.

Since a formulation thus obtained is safe and less toxic, it can be administered to a human or a mammalian animal (for example, rat, mouse, rabbit, sheep, swine, cattle, cat, dog, monkey and the like).

While a receptor protein of the invention may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

While a DNA of the invention may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(3) Gene Diagnostic Agent

Since a DNA of the invention, when used as a probe, can detect an abnormality (gene abnormality) in a DNA or an mRNA encoding a protein or its partial peptide of the invention in a human or a mammalian animal (for example, rat, mouse, rabbit, sheep, swine, cattle, cat, dog, monkey and the like), it is useful in a gene diagnosis of an impairment, a mutation or a reduced expression of such DNA or mRNA as well as an increase or an increased expression of such DNA or mRNA.

A gene diagnosis employing a DNA of the invention can be performed for example by a northern hybridization known per se or a PCR—SSCP method (Genomics, Vol.5, p874-879 (1989)) or a method described in Proceedings of the National Academy of Sciences of USA, Vol.86, p2766-2770 (1989).

(4) Method for Screening Compound Capable of Altering Expression Level of Receptor Protein of the Invention or a Partial Peptide Thereof

A DNA of the invention, when used as a probe, can be applied to a screening for a compound which alters the expression level of a receptor protein of the invention or a partial peptide thereof.

Thus, the invention provides a method for screening for a compound which alters the expression level of a receptor protein of the invention or a partial peptide thereof, for example, by determining the level of an mRNA of the receptor protein of the invention or the partial peptide thereof contained in (i) [1] a blood, [2] a certain organ and [3] a tissue or a cell isolated from an organ of a non-human mammal or in (ii) a transformant and the like.

Concretely, the level of an mRNA of a receptor protein of the invention or a partial peptide thereof is determined as follows.
(i) A normal or a disease-bearing non-human mammalian model animal (for example, mouse, rat, rabbit, sheep, swine, cattle, cat, dog, monkey and the like, more specifically, dementia rat, obesity mouse, arteriosclerosis rabbit, bile cancer mouse and the like) is treated with an agent (for example, anti-dementia agent, hypotensive agent, anti-cancer agent, anti-obesity agent and the like) or subjected to a mechanical stress (for example, flooding stress, electric shock, dark/light cycle, low temperature and the like), and after a certain time period, a blood, a certain organ (for example, brain, liver, kidney and the like) or a tissue isolated from an organ or a cell is collected.

The mRNA contained in a receptor protein of the invention or a partial peptide thereof contained in a cell collected as described above can be quantified by extracting the mRNA from the cell by a standard method followed for example by TacManPCR, or can be analyzed by a Northern blotting using a means known per se.
(ii) A transformant which expresses a receptor protein of the invention or a partial peptide thereof is prepared according to the method described above and a mRNA of the receptor protein of the invention or the partial peptide thereof contained in this transformant can be quantified and analyzed similarly.

A screening for a compound altering the expression level of a receptor protein of the invention or a partial peptide thereof can be accomplished by:
(i) administering to a normal or disease-bearing non-human mammalian model animal a test compound at a certain time point before (30 minutes to 24 hours, preferably 30 minutes to 12 hours, more preferably 1 hour to 6 hours before) or after (30 minutes to 3 days, preferably 1 hour to 2 days, more preferably 1 hour to 24 days after) the exposure to an medication or a mechanical stress, or simultaneously with a medication or a mechanical stress, followed by quantifying and analyzing a mRNA of the receptor protein of the invention or the partial peptide thereof contained in a cell at a certain time point after (30 minutes to 3 days, preferably 1 hour to 2 days, more preferably 1 hour to 24 days after) the administration;
(ii) adding a test compound to a culture medium in which a transformant is cultivated by a standard method followed by quantifying and analyzing a mRNA of the receptor protein of the invention or the partial peptide thereof contained in the transformant at a certain time point after (1 day to 7 days, preferably 1 day to 3 days, more preferably 2 days to 3 days after) initiation of the cultivation.

A compound or a salt thereof obtained by a screening method of the invention is a compound having an ability of altering the expression level of a receptor protein of the invention or a partial peptide thereof, and concretely it is (a) a compound which enhances a G protein-conjugating receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) by increasing the expression level of a receptor protein of the invention or a partial peptide thereof, and (b) a compound which suppresses such cell stimulating activity by reducing the expression level of a receptor protein of the invention or a partial peptide thereof.

Such a compound may for example be a peptide, a protein, a non-peptide compound, a synthetic compound, a fermentation product and the like, which may be novel compounds or known compounds.

A compound enhancing such a cell stimulating activity is useful as a safe and less toxic pharmaceutical for enhancing a physiological activity of a receptor protein of the invention.

A compound suppressing such a cell stimulating activity is useful as a safe and less toxic pharmaceutical for reducing a physiological activity of a receptor protein of the invention.

When a compound or a salt thereof obtained by a screening method of the invention is used in a pharmaceutical compositions, a standard procedure may be followed. For example, similarly to a pharmaceutical composition containing a receptor protein of the invention, a dosage form such as tablet, capsule, elixir, microcapsule, aseptic solution or suspension can be formulated.

Since a formulation thus obtained is safe and less toxic, it can be administered to a human or a mammalian animal (for example, rat, mouse, rabbit, sheep, swine, cattle, cat, dog, monkey and the like).

While such compound may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(5) Prophylactic and/or Therapeutic Composition Against Various Diseases Containing Compound Capable of Altering Expression Level of a Receptor Protein of the Invention or a Partial Peptide Thereof

As described above, a receptor protein of the invention is considered to play some important in vivo role for example in a central nervous function. Accordingly, a compound altering the expression level of a receptor protein of the invention or a partial peptide thereof can be used as a prophylactic and/or therapeutic agent against a disease related to the dysfunction of the receptor protein of the invention.

When such compound is used as a prophylactic and/or therapeutic agent against a disease related to the dysfunction of a receptor protein of the invention, a standard formulation procedure may be followed.

For example, such compound may be given orally as an optionally sugar-coated tablet, capsule, elixir, microcapsule and the like, or parenterally as a formulation for injection such as an aseptic solution or suspension in water or pharmacological acceptable liquid. For example, such formulation can be produced by mixing the compound with a known physiologically acceptable carrier, flavor, excipient, vehicle, preservative stabilizer, binder and the like in a unit dosage form which is acceptable generally in a pharmaceutical practice. The amount of an active ingredient in such formulation should be adjusted to achieve a suitable dose within a specified range.

An additive which may be incorporated into a tablet or a capsule may for example be a binder such as gelatin, corn starch, tragacanth, gum arabic and the like, an excipient such as crystalline cellulose, an expander such as corn starch, gelatin, alginic acid and the like, a lubricant such as magnesium stearate, a sweetener such as sucrose, lactose or saccharin, a flavor such as peppermint, oil of Geultheria ovatifolia spp., cherry and the like. When a unit dosage form is a capsule, a liquid carrier such as a fat may further be incorporated in addition to the materials described above. An aseptic formulation for injection can be prepared in accordance with an ordinary pharmaceutical practice such as a dissolution or a suspension of an active ingredient, a naturally-occurring vegetable oil such as sesame oil and palm oil in a vehicle such as a water for injection. An aqueous liquid for injection may for example be physiological saline, an isotonic solution containing glucose or other auxiliary agents (for example, D-sorbitol, D-mannitol, sodium chloride) and the like, which may be used in combination with an suitable solubilizer such as an alcohol (for example, ethanol), a polyalcohol (for example, propylene glycol, polyethylene glycol), a non-ionic surfactant (for example, polysorbate 80™, HCO-50). An oily liquid may for example be sesame oil and soybean oil, which may be used in combination with an solubilizer such as benzyl benzoate, benzyl alcohol and the like.

In addition, a prophylactic and/or therapeutic agent described above may be supplemented also with a buffer agent (for example, phosphate buffer, sodium acetate buffer), an analgesic (for example, benzalkonium chloride, procaine hydrochloride), a stabilizer (for example, human serum albumin, polyethylene glycol), a preservative (for example, benzyl alcohol, phenol), an antioxidant. A formulation for injection thus prepared is then filled usually in a suitable ampoule.

Since a formulation thus obtained is safe and less toxic, it can be administered to a human or a mammalian animal (for example, rat, mouse, rabbit, sheep, swine, cattle, cat, dog, monkey and the like).

While such compound or a salt thereof may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(6) Method for Quantifying a Ligand for G Protein-Conjugating Receptor Protein of the Invention

Since a receptor protein of the invention has a binding affinity to a ligand, it can quantify an in vivo ligand concentration at a high sensitivity.

A quantification method of the invention can be employed for example in combination with a competitive method. Thus, a sample is brought into contact with a receptor of the invention and a like to determine the concentration of a ligand in the sample. Concretely, a method described in literatures [1] and [2] shown below or an analogous method may be employed.

[1] Ed. by H. Irie, “Radioimmunoassay”, (KODANSHA, 1974)

[2] Ed. by H. Irie, “Radidimmunoassay, 2nd Vol.”, (KODANSHA, 1979)

(7) Method for Screening a Compound (Agonist, Antagonist) Capable of Altering Binding Affinity Between G Protein-Conjugating Receptor Protein of the Invention and the Ligand

By using a receptor protein and the like of the invention, or by constructing an expression system of a recombinant receptor protein followed by employing a receptor binding assay system employing this expression system, a compound altering the binding affinity between a ligand and the receptor protein of the invention and the like (for example, peptide, protein, non-peptide compound, synthetic compound, fermentation product and the like) a salt thereof can efficiently been screened for.

Such a compound includes (a) a compound having a G protein-conjugating receptor-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) (so-called agonist for a receptor protein of the invention), (b) a compound having no such cell stimulating activity (so-called antagonist for a receptor protein of the invention), (c) a compound enhancing the binding affinity between a ligand and a G protein-conjugating receptor protein of the invention, or (d) a compound reducing the binding affinity between a ligand and a G protein-conjugating receptor protein of the invention (it is preferred to screening for a compound defined as (a) by a ligand determination method described above).

Thus, the invention provides a method for screening a compound or a salt thereof capable of altering the binding affinity between a ligand and a receptor protein of the invention or a partial peptide or a salt thereof, comprising comparing between (i) the case of a contact of a receptor protein of the invention or a partial peptide thereof with the ligand, and (ii) the case of a contact of the receptor protein of the invention or the partial peptide with the ligand and a test compound.

A screening method of the invention comprises determining and comparing between the levels of the binding of a ligand to such receptor protein and the cell stimulating activities upon a case (i) and upon a case (ii).

More concretely, the invention provides:
[1] a method for screening a compound or a salt thereof capable of altering the binding affinity between a ligand and a receptor protein and the like of the invention, comprising determining and comparing between the levels of the binding of a labeled ligand to the receptor protein of the invention and the like upon a contact of the labeled ligand with the receptor protein of the invention and the like, and upon a contact of the labeled ligand and a test compound with the receptor protein of the invention and the like;
[2] a method for screening a compound or a salt thereof capable of altering the binding affinity between a ligand and a receptor protein and the like of the invention, comprising determining and comparing between the levels of the binding of a labeled ligand to a cell or a membrane fraction thereof containing the receptor protein of the invention and the like upon a contact of the labeled ligand with said cell or the membrane fraction thereof, and upon a contact of the labeled ligand and a test substance with said cell or the membrane fraction thereof;
[3] a method for screening a compound or a salt thereof capable of altering the binding affinity between a ligand and a receptor protein and the like of the invention, comprising determining and comparing between the levels of the binding of a labeled ligand to the receptor protein and the like expressed on the cell membrane by cultivating a transformant containing a DNA of the invention upon a contact of the labeled ligand with such receptor protein and the like, and upon a contact of the labeled ligand and a test substance with such receptor protein;
[4] a method for screening a compound or a salt thereof capable of altering the binding affinity between a ligand and a receptor protein and the like of the invention, comprising determining and comparing between a receptor-mediated cell stimulating activities (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) upon a contact of a compound activating the receptor protein and the like (for example, a ligand for the receptor protein of the invention) with a cell containing the receptor protein and the like, and upon a contact of a compound activating the receptor protein and the like and a test compound with the cell comprising the receptor protein and the like; and,
[5] a method for screening a compound or a salt thereof capable of altering the binding affinity between a ligand and a receptor protein and the like of the invention, comprising determining and comparing between a receptor-mediated cell stimulating activities (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) upon a contact of a compound activating the receptor protein and the like (for example, a ligand for the receptor protein of the invention) with the receptor protein and the like expressed on the cell membrane by cultivating a transformant containing a DNA of the invention, and upon a contact of the compound activating the receptor protein and the like and a test compound with the receptor protein and the like expressed on the cell membrane by cultivating a transformant containing a DNA of the invention.

Before a receptor protein and the like of the invention was not available, a screening for a G protein-conjugating receptor agonist or antagonist should involve the use of a G protein-conjugating receptor protein-containing cell, tissue or cell membrane fraction, for example, of a rat to obtain a candidate compound (primary screening) followed by a test for verifying that the candidate compound surely inhibit the binding between a human protein G-conjugating receptor protein and a ligand (secondary screening). Since other receptor proteins were allowed to be present simultaneously when such cell, tissue or cell membrane was used as it is, it was difficult practically to screen for an agonist or antagonist for an intended receptor protein.

On the contrary, the use of a human receptor protein obtained according to the invention requires no such primary screening and allows a compound which inhibits the binding between a ligand and a G protein-conjugating receptor protein to be selected for at a high efficiency. In addition, a convenient judgement whether a selected compound is an agonist or an antagonist becomes possible.

A concrete screening method of the invention is described below.

A receptor protein and the like of the invention employed in a screening method of the invention may be any one containing a receptor proteins of the invention and the like described above, and is preferably a cell membrane fraction of an organ of a mammalian animal containing the receptor of the invention and the like. Nevertheless, a screening may employ a human receptor protein which is expressed in a large amount using a recombinant since an organ derived especially from a human is extremely difficult to obtain.

In order to produce a receptor protein of the invention, an expression method described above may be employed, and it is preferred to express a DNA of the invention in a mammalian cell or an insect cell. While a complementary DNA is employed as a DNA fragment encoding a target protein moiety, it is not limiting. For example, a gene fragment or a synthetic DNA may also be employed. For the purpose of introducing a DNA fragment encoding a receptor protein of the invention into an animal host cell to effect a highly efficient expression, it is preferred to integrate such DNA fragment into the downstream of a polyhedron promoter of a nuclear polyhedrosis virus (NPV) classified as a vaculovirus whose host is an insect, an SV 40-derived promoter, a retrovirus promoter, a metallothioneine promoter, a human heat shock promoter, a cytomegalovirus promoter, an SRα promoter and the like. An expressed receptor can be examined for its quantity or quality by a method known per se. For example, a method known in a literature (Nambi, P. et al., J.Biol.Chem., Vo.267, p.19555 to 19559, 1992) may be employed.

Accordingly, in a screening method of the invention, those containing a receptor protein and the like of the invention may be a receptor protein and the like which was purified by a method known per se or a cell containing such receptor protein or a cell membrane fraction containing such receptor protein.

When a cell containing a receptor protein of the invention is employed in a screening method of the invention, this cell may be immobilized by glutaraldehyde or formalin. Such immobilization can be effected by a method known per se.

A cell containing a receptor protein of the invention is a host cell expressing the receptor protein of the invention, and such host cell may preferably be an Escherichia, a Bacillus, an yeast, an insect cell, an animal cell and the like.

A cell membrane fraction means a cell membrane-rich fraction obtained by a method known per se after pelletizing the cell. A cell may be pelletized for example by a method in which a cell is pressed and crashed by a Potter-Elvehjem homogenizer, by using a whirling blender or a polytron (Kinematica), by means of an ultrasonic treatment, or by a method in which a cell is sprayed via a fine nozzle while being pressurized by a French press. A cell membrane may be fractionated mainly by a centrifugal fractionation such as a fractional centrifugation or a density gradient centrifugation. For example, a cell pellet is centrifuged at a low speed (500 rpm to 3000 rpm) for a short period (usually about 1 minutes to 10 minutes) to obtain a supernatant, which is then centrifuged at a higher speed (15000 rpm to 30000 rpm) usually for 30 minutes to 2 hours to obtain a pellet, which is used as a membrane fraction. This membrane fraction contains a large amount of the membrane components such as an expressed receptor protein and phospholipids and membrane proteins derived from the cell.

The amount of a receptor protein in a cell containing such receptor protein or a membrane fraction thereof is preferably 10³ to 10⁸ molecules, more preferably 10⁵ to 10⁷ molecules per cell. A higher expression leads to a higher ligand binding activity (specific activity) per membrane fraction, whereby allowing not only a highly sensitive screening system to be established but also a large amount of a sample to be determined in an identical lot.

For performing the methods (1) to (3) described above for screening for a compound altering the binding affinity between a ligand and a receptor protein and the like of the invention, a suitable receptor protein fraction and a labeled test compound are required.

A receptor protein fraction is preferably a natural receptor protein fraction or a recombinant receptor fraction having an activity which is equivalent to that of the natural one. The expression “activity which is equivalent” employed here means an equivalent ligand binding activity or signal transmission activity.

A labeled ligand may be a labeled ligand and a labeled ligand analogue compound. For example, a ligand labeled with [³H], [¹²⁵I], [¹⁴C] or [³⁵S] may be employed.

Concretely, for performing a method for screening a for a compound altering the binding affinity between a ligand and a receptor protein and the like of the invention, a cell containing the receptor protein of the invention or a membrane fraction of the cell is suspended in a buffer suitable for the determination method to prepare a receptor protein standard. Such buffer may for example be a buffer which is not inhibit the binding between a ligand and the receptor protein, such as a phsophate buffer or tris-HCl buffer, pH 4 to 10 (preferably pH 6 to 8). Also for the purpose of reducing the non-specific binding, a surfactant such as CHAPS, Tween-80™ (KAO-ATLAS), digitonin, deoxycholate and the like or various protein such as bovine serum albumin and gelatin may be added to a buffer. In addition, for the purpose of suppressing the cleavage of a receptor or a ligand by a protease, a protease inhibitor such as PMSF, leupeptine, E-64 (PEPTIDE KENKYUSHO) and a pepstatin may also be added. In a 0.01 ml to 10 ml of a solution of the receptor described above, a certain amount (5000 cpm to 500000 cpm) of a labeled ligand is added and a test compound is allowed to be present simultaneously at a concentration of 10⁻⁴M to 10⁻¹⁰M. For measuring the non-specific binding (NSB) level, a reaction tube containing a large excess of the non-labeled ligand may also be provided. The reaction is performed at a temperature of about 0° C. to 50° C., preferably about 4° C. to 37° C. for a period of about 20 minutes to 24 hours, preferably about 30 minutes to 3 hours. After the reaction, the mixture is filtered through a filter such as a glass fiber filter paper and washed with an appropriate volume of the same buffer and then the radioactivity remaining on the glass fiber filter paper is determined by a liquid scintillation counter or a gamma-counter. With regarding the count (B₀—NSB) obtained by subtracting the non-specific binding (NSB) level from the binding (B₀) in the absence of any competitive substance as 100%, a compound whose specific binding (B—NSB) is 50% or less can be selected as a candidate having a competitive inhibitory effect.

In order to perform the methods [4] to [5] described above for screening for a compound altering the binding affinity between a ligand and a receptor protein and the like of the invention, such receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) can be determined by a known method or using a commercial assay kit.

Concretely, a cell containing a receptor protein and the like of the invention is cultivated in a multi-well plate. For a screening, the medium is replaced with a fresh buffer or an appropriate buffer which is non-toxic to the cell, and a test compound is added and incubated for a certain period, and then the cell is extracted or the supernatant is recovered and the product accumulated is quantified by a relevant method. When the production of a substance which is an index for a cell stimulating activity (for example, arachidonic acid) is difficult to be assayed due to any degrading enzyme contained in the cell, an inhibitor of such degrading enzyme may be added to perform the assay. Another activity such as a cAMP production inhibiting activity can be detected as a production inhibiting effect in a cell whose basal production has been increased using forskolin and the like.

To perform a screening by determining a cell stimulating activity, a suitable cell expressing a receptor protein is required. A cell expressing a receptor protein and the like of the invention is desirably be a cell line having a natural receptor protein and the like of the invention and a cell line expressing a recombinant receptor protein and the like described above.

A test compound may be a peptide, a protein, a non-peptide compound, a synthetic compound, a fermentation product, a cell extract, a plant extract, an animal tissue extract and the like, which may be novel compound or known compound.

A kit of the invention for screening a compound altering the binding affinity between a ligand and a receptor protein and the like of the invention comprises the receptor protein and the like of the invention, a cell containing the receptor protein and the like of the invention or a membrane fraction of a cell containing the receptor protein and the like of the invention.

A screening kit of the invention may for example be one of those listed below.

1. Reagents for Ligand Determination

[1] Assay Buffer and Washing Buffer

Hanks' Balanced Salt Solution (Gibco) supplemented with 0.05% bovine serum albumin (Sigma).

The buffer is sterilized by filtering through a filter whose pore size is 0.45 μm, and may be stored at 4° C. or prepared just before use.

[2] Preparation of G Protein-Conjugating Receptor

A CHO cell expressing a receptor protein of the invention is subjected to a subculture in a 12-well plate at the density of 5×10⁵ cells/well and cultivated at 37° C. under 5% CO₂ and 95% air for 2 days.

[3] Labeled Test Compound

A commercially available [³H]—, [125I]—, [¹⁴C]— or [³⁵S]-labeled ligand.

A ligand in an aqueous solution is stored at 4° C. or −20° C. and diluted at 1 μM with an assay buffer just before use.

[4] Ligand Standard Solution

A ligand is dissolved at 1 mM with a PBS supplemented with 0.1% bovine serum albumin (Sigma) and stored at −20° C.

2. Assay Procedures

[1] An inventive receptor protein-expressing CHO cell which has been cultivated in a 12-well tissue culture plate is washed twice with 1 ml of an assay buffer and each 490 μM of the assay buffer is added to each well.

[2] After adding 5 μl of a 10⁻³ to 10⁻¹⁰ M solution of a test compound, 5 μl of a labeled ligand is added and allowed to react at room temperature for 1 hour. 5 μl of a 10⁻³ ligand is added instead of the test compound for measuring the non-specific binding.

[3] The reaction mixture is removed and the cell is washed three times with 1 ml of the washing buffer. The labeled ligand bound to the cell is dissolved by 0.2 N NaOH-1% SDS, and mixed with 4 ml of a liquid scintillator A (Wako Pure Chemical).

[4] Using a liquid scintillation counter (Beckman), the radioactivity is determined, and a percent maximum binding (PMB) is calculated as follows.
PMB=[(B−NSB)/(B₀−NSB)]×100
wherein PMB is a percent maximum binding, B is a binding level in the presence of a sample, NSB is a non-specific binding level and B₀ is the maximum binding level.

A compound or a salt which can be obtained by a screening method or using a screening kit of the invention is a compound having an ability of altering the binding affinity between a ligand and a receptor protein and the like of the invention, and concretely it is (a) a compound having a G protein-conjugating receptor-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) (so-called agonist for a receptor protein of the invention), (b) a compound having no such cell stimulating activity (so-called antagonist for a receptor protein of the invention), (c) a compound enhancing the binding affinity between a ligand and a G protein-conjugating receptor protein of the invention, or (d) a compound reducing the binding affinity between a ligand and a G protein-conjugating receptor protein of the invention.

Such a compound may for example be a peptide, a protein, a non-peptide compound, a synthetic compound, a fermentation product and the like, which may be novel compounds or known compounds.

Since an agonist for a receptor protein and the like of the invention has a physiological effect similar to that possessed by a ligand for the receptor protein and the like of the invention, it is useful as a safe and less toxic pharmaceutical utilizing the relevant ligand activity.

Since an antagonist for a receptor protein and the like of the invention has an inhibitory effect on the physiological effect of a ligand for the receptor protein and the like of the invention, it is useful as a safe and less toxic pharmaceutical inhibiting the ligand activity.

A compound enhancing the binding affinity between a ligand and a G protein-conjugating receptor protein of the invention is useful as a safe and less toxic pharmaceutical for enhancing the bioactivity of a ligand for the receptor protein and the like of the invention.

A compound reducing the binding affinity between a ligand and a G protein-conjugating receptor protein of the invention is useful as a safe and less toxic pharmaceutical for reducing the bioactivity of a ligand for the receptor protein and the like of the invention.

When a compound or a salt thereof obtained by a screening method or using a screening kit of the invention is used in a pharmaceutical compositions, a standard procedure may be followed. For example, similarly to a pharmaceutical composition containing a receptor protein of the invention, a dosage form such as tablet, capsule, elixir, microcapsule, aseptic solution or suspension can be formulated.

Since a formulation thus obtained is safe and less toxic, it can be administered to a human or a mammalian animal (for example, rat, mouse, rabbit, sheep, swine, cattle, cat, dog, monkey and the like).

While such compound may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(8) Prophylactic and/or Therapeutic Composition Against Various Diseases Containing Compound (Agonist, Antagonist) Capable of Altering the Binding Affinity Between G Protein-Conjugating Receptor Protein of the Invention and Ligand

As described above, a receptor protein of the invention is considered to play some important in vivo role for example in a central nervous function. Accordingly, a compound (agonist, antagonist) capable of altering the binding affinity between a receptor protein of the invention and a ligand can be used as a prophylactic and/or therapeutic agent against a disease related to the dysfunction of the receptor protein of the invention.

When such compound is used as a prophylactic and/or therapeutic agent against a disease related to the dysfunction of a receptor protein of the invention, a standard procedure may be followed.

For example, such compound may be given orally as an optionally sugar-coated tablet, capsule, elixir, microcapsule and the like, or parenterally as a formulation for injection such as an aseptic solution or suspension in water or pharmacological acceptable liquid. For example, such formulation can be produced by mixing the compound with a known physiologically acceptable carrier, flavor, excipient, vehicle, preservative stabilizer, binder and the like in a unit dosage form which is acceptable generally in a pharmaceutical practice. The amount of an active ingredient in such formulation should be adjusted to achieve a suitable dose within a specified range.

An additive which may be incorporated into a tablet or a capsule may for example be a binder such as gelatin, corn starch, tragacanth, gum arabic and the like, an excipient such as crystalline cellulose, an expander such as corn starch, gelatin, alginic acid and the like, a lubricant such as magnesium stearate, a sweetener such as sucrose, lactose or saccharin, a flavor such as peppermint, oil of Geultheria ovatifolia spp., cherry and the like. When a unit dosage form is a capsule, a liquid carrier such as a fat may further be incorporated in addition to the materials described above. An aseptic formulation for injection can be prepared in accordance with an ordinary pharmaceutical practice such as a dissolution or a suspension of an active ingredient, a naturally-occurring vegetable oil such as sesame oil and palm oil in a vehicle such as a water for injection. An aqueous liquid for injection may for example be physiological saline, an isotonic solution containing glucose or other auxiliary agents (for example, D-sorbitol, D-mannitol, sodium chloride) and the like, which may be used in combination with an suitable solubilizer such as an alcohol (for example, ethanol), a polyalcohol (for example, propylene glycol, polyethylene glycol), a non-ionic surfactant (for example, polysorbate 80™, HCO-50). An oily liquid may for example be sesame oil and soybean oil, which may be used in combination with an solubilizer such as benzyl benzoate, benzyl alcohol and the like.

In addition, a prophylactic and/or therapeutic agent described above may be supplemented also with a buffer agent (for example, phosphate buffer, sodium acetate buffer), an analgesic (for example, benzalkonium chloride, procaine hydrochloride), a stabilizer (for example, human serum albumin, polyethylene glycol), a preservative (for example, benzyl alcohol, phenol), an antioxidant. A formulation for injection thus prepared is then filled usually in a suitable ampoule.

Since a formulation thus obtained is safe and less toxic, it can be administered to a human or a mammalian animal (for example, rat, mouse, rabbit, sheep, swine, cattle, cat, dog, monkey and the like).

While such compound or a salt there of may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(9) Quantification of a Receptor Protein of the Invention or Partial Peptide Thereof or Salt thereof

Since an antibody of the invention can recognize the receptor protein and the like of the invention specifically, it can be used for quantifying the receptor protein and the like of the invention in a test sample, especially by a sandwich immunoassay. Thus, the present invention also provides, for example:
(i) a method for quantifying a receptor protein and the like of the invention in a test sample, comprising reacting competitively an antibody of the invention with the test sample and a labelled receptor protein and the like of the invention followed by a determination of the ratio of the labelled receptor protein and the like of the invention bound to the antibody, and,
(ii) a method for quantifying a receptor protein and the like of the invention in a test sample, comprising reacting simultaneously or sequentially the test sample with an antibody of the invention insolubilized on a carrier and a labelled other antibody of the invention followed by a determination of the activity of the label on the insolubilized carrier.

In the method of (ii) described above, it is preferred that one antibody is an antibody recognizing the N-terminal of a receptor protein and the like of the invention and the other is reactive with the C-terminal of the protein of the invention.

Using a monoclonal antibody against a receptor protein and the like of the invention (hereinafter sometimes referred to as a monoclonal antibody of the invention), the receptor protein and the like of the invention can be quantified and a detection by a tissue staining and the like can also be performed. For these purposes, an antibody molecule itself may be used, or a F(ab′)₂, Fab′ or Fab fraction of the antibody molecule may also be employed. A method for quantifying a receptor protein and the like of the invention using an antibody of the invention is not particularly limited, and may be any method in which the amount of an antibody, antigen or antibody-antigen complex corresponding to the amount of the antigen (for example, the amount of the receptor protein) in a test sample is detected physically and then a calculation is made based on a standard curve obtained by using the standard solutions containing known amounts of the antigen. For example, a nephelometry, a competitive assay, an immunometric assay and a sandwich assay can preferably be employed, with a sandwich assay described below being preferred in view of the sensitivity and the specificity.

A label employed in an assay using a labelled substance may for example be a radioisotope, an enzyme, a fluorescent substance, a luminescent substance and the like. Such radioisotope may for example be [125I], [131I], [³H], [¹⁴C] and the like. An enzyme described above is preferably one which is stable and has a high specific activity and may for example be β-galactosidase, β-glucosidase, alkaline phosphatase, peroxidase, malate dehydrogenase and the like. A fluorescent substance may for example be fluorescamine, fluorescein isothiocyanate and the like. A luminescent substance may for example be luminol, a luminol derivative, luciferin, lucigenin and the like. A biotin-avidin system may also be employed for binding a label to an antibody or an antigen.

An insolubilization of an antigen or an antibody may be effected utilizing a physical adsorption, and a chemical binding employed usually for insolubilizing a protein or an enzyme may also be employed. A carrier may for example be an insoluble polysaccharide such as agarose, dextran and cellulose, a synthetic resin such as polystyrene, polyacrylamide and silicon, as well as a glass.

In a sandwich method, an insolubilized inventive monoclonal antibody is reacted with a test sample (primary reaction), and then a labelled other monoclonal antibody of the invention is reacted (secondary reaction), and then the activity of the label on the insolubilized carrier is determined, whereby quantifying a receptor protein of the invention in the test sample. The primary reaction and the secondary reaction can be performed in the opposite order or may be performed simultaneously or at an interval. A label and a method for the insolubilization are as described above.

It is not necessary always in a sandwich immunoassay that an antibody employed as an antibody for a solid phase or an antibody for labeling is of a single type, and not less than two antibodies can be employed as in a mixture for the purpose of a higher sensitivity of the measurement.

In a method for determining a receptor protein and the like by a sandwich method of the invention, monoclonal antibodies of the present invention employed in the primary reaction and the secondary reaction are preferably those differing from each other in the site of the binding to the receptor protein and the like. That is, the antibody employed in the primary and secondary reactions are selected so that when the antibody employed in the secondary reaction recognizes the C-terminal of the receptor protein then the antibody employed in the primary reaction recognizes the sites other than the C-terminal, such as the N-terminal.

A monoclonal antibody of the present invention can be used in an assay system other than a sandwich assay, such as a competitive assay, an immunometric assay and a nephelometry. In a competitive assay, an antigen in a test sample and a labelled antigen are reacted competitively with an antibody and then unreacted labelled antigen (F) is separated from an antibody-binding labelled antigen (B) (B/F separation), and the amount of the label on either B or F is determined, whereby quantifying the antigen in the test sample. This reaction is conducted by a liquid phase method employing a soluble antibody as an antibody and performing a B/F separation using a polyethylene glycol and a secondary antibody, and also by a solid phase method employing a solid phase antibody as a primary antibody or employing a soluble antibody as a primary antibody and a solid phase antibody as a secondary antibody.

In an immunometric method, an antigen in a test sample and a solid phase antigen are reacted competitively with a certain amount of a labelled antibody, and then the solid phase is separated from a liquid phase, or an antigen in a test sample is reacted with a labelled antibody in excess and then a solid phase antigen is added to bind an unreacted labelled antibody to the solid phase and subsequently the solid phase is separated from a liquid phase. Then the amount of the label in either phase is determined, whereby quantifying the antigen in the test sample.

In an nephelometric assay, the amount of an insoluble precipitate formed as a result of an antigen-antibody reaction in a gel or a solution is determined. Even when the amount of an antigen in a test sample is very small and only a small amount of a precipitation can be obtained, a laser nephelometry utilizing a scattering of the laser is employed preferably.

When applying each immunological assay described above to a quantification method of the invention, no particular condition or operation is specified. Ordinary conditions and operations in each method may be employed in combination with a technology known by those skilled in the art to construct an assay system for a receptor protein of the invention or a salt thereof. Such general technology means is found in corresponding textbooks or guidebooks [for example, “Radioimmunoassay”, ed. by H. Irie (KODANSHA, 1974), “Radioimmunoassay II”, ed. by H. Irie (KODANSHA, 1979), “Enzyme Immunoassay”, ed. by E. Ishikawa et al., (IGAKUSHOIN, 1978), “Enzyme Immunoassay”, (2nd Volume), ed. by E. Ishikawa et al., (IGAKUSHOIN, 1982), “Enzyme Immunoassay”, (3rd Volume), ed. by E. Ishikawa et al., (IGAKUSHOIN, 1987), “Methods in Enzymology”, Vol.70, idem (Immunochemical Techniques (Part A)), Vol.73, idem (Immunochemical Techniques (Part B)), Vol.74, idem (Immunochemical Techniques (Part C)), Vol.84, idem (Immunochemical Techniques (Part D: Selected Immunoassays)), Vol.92, idem (Immunochemical Techniques (Part E: Monoclonal Antibodies and General Immunoassay Methods)), Vol.121, idem (Immunochemical Techniques (Part I: Hybridoma Technology and Monoclonal Antibodies)), (Academic Press)].

As described above, a receptor protein of the invention or a salt thereof can be quantified at a high sensitivity by employing an antibody of the invention.

Furthermore, by quantifying a receptor protein of the invention or a salt thereof using an antibody of the invention, various diseases related to the dysfunction of the receptor protein of the invention can be diagnosed.

An antibody of the invention can also be employed for a specific detection a receptor protein and the like of the invention which is present in a test sample such as a body fluid or a tissue. It can also be employed in preparing an antibody column used for purifying a receptor protein and the like of the invention, in detecting a receptor protein and the like of the invention in each fraction upon a purification, or in analyzing the behavior of a receptor protein and the like of the invention in a test cell.

(10) Method for Screening Compound Capable of Altering Amount of Receptor Protein of the Invention or Partial Peptide thereof in Cell Membrane

Since an antibody of the invention can recognize a receptor protein or a partial peptide or a salt thereof specifically, it can be used for screening for a compound altering the amount of the receptor protein or the partial peptide or the salt of the invention in a cell membrane.

Thus, the present invention provides, for example:
(i) a method for screening for a compound altering the amount of a receptor protein of the invention or a partial peptide thereof contained in a cell membrane fraction by destroying tissue or cell isolated from [1] a blood, [2] a certain organ or [3] a tissue of a non-human mammalian animal, and isolating a cell membrane fraction, and then quantifying the receptor protein of the invention or the partial peptide thereof contained in the cell membrane fraction;
(ii) a screening method for a compound altering the amount of a receptor protein of the invention or a partial peptide thereof in a cell membrane by destroying a transformant expressing the receptor protein of the invention or the partial peptide thereof, and isolating a cell membrane fraction, and then quantifying the receptor protein of the invention or the partial peptide thereof contained in the cell membrane fraction;
(iii) a method for screening for a compound altering the amount of a receptor protein of the invention or a partial peptide thereof in a cell membrane by making [1] a blood, [2] a certain organ or [3] a tissue or a cell isolated from an organ of a non-human mammalian animal into sections, and using an imminostaining method to quantify the staining of the receptor protein in the surface layer of the cell, whereby identifying the protein on the cell membrane.

The present invention also provides:
(iv) a screening method for a compound altering the amount of a receptor protein of the invention or a partial peptide thereof in a cell membrane by making a transformant expressing the receptor protein of the invention or the partial peptide thereof into sections, and using an imminostaining method to quantify the staining of the receptor protein in the surface layer of the cell, whereby identifying the protein on the cell membrane.

Concretely, the level of a receptor protein of the invention or a partial peptide thereof contained in a cell membrane fraction is quantified as follows.
(i) A normal or a disease-bearing non-human mammalian model animal (for example, mouse, rat, rabbit, sheep, swine, cattle, cat, dog, monkey and the like, more specifically, dementia rat, obesity mouse, arteriosclerosis rabbit, bile cancer mouse and the like) is treated with an agent (for example, anti-dementia agent, hypotensive agent, anti-cancer agent, anti-obesity agent and the like) or subjected to a mechanical stress (for example, flooding stress, electric shock, dark/light cycle, low temperature and the like), and after a certain time period, a blood, a certain organ (for example, brain, liver, kidney and the like) or a tissue or isolated from an organ a cell is collected. The organ, tissue or cell thus obtained is suspended in a suitable buffer solution (for example, Tris-HCl buffer, phosphate buffer, Hepes buffer) where it is destroyed and then exposed to a surfactant (for example Triton X100™, Tween 20™) and then subjected to a centrifugation, a filtration or a column fractionation, whereby obtaining a cell membrane fraction.

A cell membrane fraction means a cell membrane-rich fraction obtained by a method known per se after pelletizing the cell. A cell may be pelletized for example by a method in which a cell is pressed and crashed by a Potter-Elvehjem homogenizer, by using a whirling blender or a polytron (Kinematica), by means of an ultrasonic treatment, or by a method in which a cell is sprayed via a fine nozzle while being pressurized by a French press. A cell membrane may be fractionated mainly by a centrifugal fractionation such as a fractional centrifugation or a density gradient centrifugation. For example, a cell pellet is centrifuged at a low speed (500 rpm to 3000 rpm) for a short period (usually about 1 minutes to 10 minutes) to obtain a supernatant, which is then centrifuged at a higher speed (15000 rpm to 30000 rpm) usually for 30 minutes to 2 hours to obtain a pellet, which is used as a membrane fraction. This membrane fraction contains a large amount of the membrane components such as an expressed receptor protein and phospholipids and membrane proteins derived from the cell.

A receptor protein or a partial peptide of the invention contained in a cell membrane fraction can be quantified for example by a sandwich immunoassay employing an antibody of the invention as well as a Western blotting.

Such sandwich immunoassay can be performed similarly to a method described above, while the western blotting can be performed by a method known per se.
(ii) A transformant which expresses a receptor protein of the invention or a partial peptide thereof is prepared according to the method described above and the receptor protein of the invention or the partial peptide thereof contained in a cell membrane fraction can be quantified.

A screening for a compound altering the level of a receptor protein of the invention or a partial peptide thereof in a cell membrane can be accomplished by:
(i) administering a test compound at a certain time point before (30 minutes to 24 hours, preferably 30 minutes to 12 hours, more preferably 1 hour to 6 hours before) or at a certain time point after (30 minutes to 3 days, preferably 1 hour to 2 days, more preferably 1 hour to 24 days after) subjecting a normal or disease-bearing non-human mammalian model animal to a medication or a mechanical stress, or simultaneously with a medication or a mechanical stress, followed by quantifying and analyzing the receptor protein of the invention or the partial peptide thereof contained in a cell at a certain time point after (30 minutes to 3 days, preferably 1 hour to 2 days, more preferably 1 hour to 24 days after) the administration;
(ii) adding a test compound to a culture medium in which a transformant is cultivated by a standard method followed by quantifying and analyzing the receptor protein of the invention or the partial peptide thereof contained in a cell membrane at a certain time point after (1 day to 7 days, preferably 1 day to 3 days, more preferably 2 days to 3 days after) initiation of the cultivation.

Concretely, the level of a receptor protein of the invention or a partial peptide thereof contained in a cell membrane fraction is determined as follows.
(iii) A normal or a disease-bearing non-human mammalian model animal (for example, mouse, rat, rabbit, sheep, swine, cattle, cat, dog, monkey and the like, more specifically, dementia rat, obesity mouse, arteriosclerosis rabbit, bile cancer mouse and the like) is treated with an agent (for example, anti-dementia agent, hypotensive agent, anti-cancer agent, anti-obesity agent and the like) or subjected to a mechanical stress (for example, flooding stress, electric shock, dark/light cycle, low temperature and the like), and after a certain time period, a blood, a certain organ (for example, brain, liver, kidney and the like) or a tissue isolated from an organ or a cell is collected. The organ, tissue or cell thus obtained is cut into sections by a standard method, and subjected to a immunostaining using an antibody of the invention. By quantifying the staining of the receptor protein in the surface layer of the cell to identify the protein on the cell membrane, a verification of the level of the receptor protein of the invention or the partial peptide thereof in the cell membrane is possible.
(iv) A verification is possible also by subjecting a transformant expressing a receptor protein or a salt thereof to the similar procedure.

A compound or a salt thereof obtained by a screening method of the invention is a compound having an ability of altering the level of a receptor protein of the invention or a partial peptide thereof in a cell membrane, and concretely it is (a) a compound which enhances the G protein-conjugating receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) by increasing the level of a receptor protein of the invention or a partial peptide thereof in a cell membrane, and (b) a compound which suppresses such cell stimulating activity by reducing the level of a receptor protein of the invention or a partial peptide thereof.

Such a compound may for example be a peptide, a protein, a non-peptide compound, a synthetic compound, a fermentation product and the like, which may be novel compounds or known compounds.

A compound enhancing such a cell stimulating activity is useful as a safe and less toxic pharmaceutical for enhancing a physiological activity of a receptor protein of the invention.

A compound suppressing such a cell stimulating activity is useful as a safe and less toxic pharmaceutical for reducing a physiological activity of a receptor protein of the invention.

When a compound or a salt thereof obtained by a screening method of the invention is used in a pharmaceutical compositions, a standard procedure may be followed. For example, similarly to a pharmaceutical composition containing a receptor protein of the invention, a dosage form such as tablet, capsule, elixir, microcapsule, aseptic solution or suspension can be formulated.

Since a formulation thus obtained is safe and less toxic, it can be administered to a human or a mammalian animal (for example, rat, mouse, rabbit, sheep, swine, cattle, cat, dog, monkey and the like).

While such compound may be given at various doses depending on the patient to be treated, the target organ, the condition of the patient and the administration route, it can be given orally to an infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(11) Prophylactic and/or Therapeutic Composition Against Various Diseases Containing Compound Capable of Altering Amount of Receptor Protein of the Invention or Partial peptide thereof in Cell Membrane

As described above, a receptor protein of the invention is considered to play some important in vivo role for example in a central nervous function. Accordingly, a compound altering the expression level of a receptor protein of the invention or a partial peptide thereof in a cell membrane can be used as a prophylactic and/or therapeutic agent against a disease related to the dysfunction of the receptor protein of the invention.

When such a compound is used as a prophylactic and/or therapeutic agent against a disease related to the dysfunction of a receptor protein of the invention, a standard formulation procedure may be followed.

For example, such compound may be given orally as an optionally sugar-coated tablet, capsule, elixir, microcapsule and the like, or parenterally as a formulation for injection such as an aseptic solution or suspension in water or pharmacological acceptable liquid. For example, such formulation can be produced by mixing the compound with a known physiologically acceptable carrier, flavor, excipient, vehicle, preservative stabilizer, binder and the like in a unit dosage form which is acceptable generally in a pharmaceutical practice. The amount of an active ingredient in such formulation should be adjusted to achieve a suitable dose within a specified range.

An additive which may be incorporated into a tablet or a capsule may for example be a binder such as gelatin, corn starch, tragacanth, gum arabic and the like, an excipient such as crystalline cellulose, an expander such as corn starch, gelatin, alginic acid and the like, a lubricant such as magnesium stearate, a sweetener such as sucrose, lactose or saccharin, a flavor such as peppermint, oil of Geultheria ovatifolia spp., cherry and the like. When a unit dosage form is a capsule, a liquid carrier such as a fat may further be incorporated in addition to the materials described above. An aseptic formulation for injection can be prepared in accordance with an ordinary pharmaceutical practice such as a dissolution or a suspension of an active ingredient, a naturally-occurring vegetable oil such as sesame oil and palm oil in a vehicle such as a water for injection. An aqueous liquid for injection may for example be physiological saline, an isotonic solution containing glucose or other auxiliary agents (for example, D-sorbitol, D-mannitol, sodium chloride) and the like, which may be used in combination with an suitable solubilizer such as an alcohol (for example, ethanol), a polyalcohol (for example, propylene glycol, polyethylene glycol), a non-ionic surfactant (for example, polysorbate 80™, HCO-50). An oily liquid may for example be sesame oil and soybean oil, which may be used in combination with an solubilizer such as benzyl benzoate, benzyl alcohol and the like.

In addition, a prophylactic or therapeutic agent described above may be supplemented also with a buffer agent (for example, phosphate buffer, sodium acetate buffer), an analgesic (for example, benzalkonium chloride, procaine hydrochloride), a stabilizer (for example, human serum albumin, polyethylene glycol), a preservative (for example, benzyl alcohol, phenol), an antioxidant. A formulation for injection thus prepared is then filled usually in a suitable ampoule.

Since a formulation thus obtained is safe and less toxic, it can be administered to a human or a mammalian animal (for example, rat, mouse, rabbit, sheep, swine, cattle, cat, dog, monkey and the like).

While such compound or a salt there of may be given at various doses depending on the patient to be treated, the target organ, the condition of the patient and the administration route, it can be given orally to an is infertility patient (60 kg) usually at a daily dose of about 0.1 mg to 100 mg, preferably about 1.0 to 50 mg, more preferably about 1.0 to 20 mg. When it is given parenterally, the single dose may vary depending on the patient to be treated, the target organ, the condition of the patient and the administration route, and may for example be given as a formulation for injection to an infertility patient (60 kg) usually at a daily dose of about 0.01 mg to 30 mg, preferably about 0.1 to 20 mg, more preferably about 0.1 to 10 mg, which is given advantageously by an intravenous injection. Also in other animals, a dose calculated for a 60 kg body weight may be administered.

(12) Neutralization of Receptor Protein of the Invention or Partial Peptide thereof or Salt Thereof by an Antibody

The neutralizing activity of an antibody against a receptor protein of the invention or a partial peptide thereof means an activity by which a signal transmission function involving such receptor protein and the like is inactivated. Accordingly, when such antibody having a neutralizing activity, a signal transmission involving such receptor protein, such as the receptor protein-mediated cell stimulating activity (for example, an activity which promotes or suppresses arachidonic acid release, acetylcholin release, intracellular Ca²⁺ release, intracellular cAMP production, intracellular cGMP production, inositol phosphate production, cell membrane potential variation, intracellular protein phosphorylation, c-fos activation, pH reduction and the like) can be inactivated. As a result, an application to the prevention and/or the treatment of a disease caused for example by an overexpression of such receptor protein is possible.

(13) Creation of Animal Having DNA encoding G Protein-Conjugating Receptor Protein of the Invention

A transgenic animal expressing a receptor protein and the like of the invention can be created using a DNA of the invention. Such an animal may for example be a mammalian animal (for example, rat, mouse, rabbit, sheep, swine, cattle, cat, dog, monkey and the like) and the like (hereinafter abbreviated as an animal), with mouse and rabbit being preferred particularly.

When a DNA of the invention is transferred to a target animal, a use as a gene construct bound downstream of a promoter capable of expressing such DNA in an animal cell is usually advantageous. When a DNA of the invention which is derived for example from a rabbit is to be transferred, a gene construct bound downstream of any promoter capable of expressing a DNA of the invention derived from an animal having a high homology thereto is microinjected for example to a fertilized ovum of a rabbit to create a DNA transferred animal producing a receptor protein and the like of the invention at a high level. While such promoter may for example be a ubiquitous expression promoter such as virus-derived promoter and metallothioneine, an NGF gene promoter expressing specifically in a brain or an enolase gene promoter are employed preferably.

The transfer of a DNA of the invention at the stage of a fertilized ovum is carried out to ensure presentation of the DNA throughout the entire embryonic and somatic cells in a target animal. The presence of a receptor protein and the like of the invention in an embryonic cell of a created animal after the DNA transfer means that the entire off-springs of the created animal will have the receptor and the like of the invention in their embryonic and somatic cells. Thus, an off-spring of this species taking over this gene will have the receptor protein and the like of the invention in its entire embryonic and somatic cells.

A DNA transferred animal of the invention, once ensuring a stable preservation of the gene even after a mating, can be subjected as a relevant DNA-possessing animal to a hereditary breeding under an ordinary breeding condition. Furthermore, by mating the animals of the both sexes each having an intended DNA, a homozygote animal having the transferred genes in the both of their homologous chromosomes can be obtained, and then by mating the animals of the both sexes thus obtained a hereditary breeding to allow all off-springs to have the relevant DNA is possible.

Since an animal to which a DNA of the invention has been transferred allows a receptor protein and the like of the invention to be expressed highly, it is useful as an animal for screening for an agonist or an antagonist for the receptor protein and the like of the invention.

A DNA transferred animal of the invention can be used also as a cell source for a tissue culture. For example, by analyzing a DNA or a RNA in a tissue of a DNA transferred mouse of the invention directly or analyzing a tissue having a receptor protein of the invention expressed, the receptor protein and the like of the invention can be investigated. A cell of a tissue having a receptor protein of the invention is cultivated by a standard tissue culture technology and used to investigate the function of a cell from a tissue which is generally difficult to be cultivated, such as a brain and a peripheral tissue. Also by using such cell, a pharmaceutical capable of promoting the function of each tissue can be selected. Furthermore, a receptor protein and the like of the invention can be isolated and purified from a highly expressing cell.

In the specification and the drawings, a base or an amino acid may be designated as a code based on IUPAC-IUB, Commission on Biological Nomenclature or as a customary abbreviation in the art as exemplified below. When an amino acid is present as an optical isomer, it is in L-form unless otherwise specified.
DNA: Deoxyribonucleic acid
cDNA: Complementary deoxyribonucleic acid
A: Adenine
T: Thymine
G: Guanine
C: Cytosine
RNA: Ribonucleic acid
mRNA: Messenger ribonucleic acid
dATP: Deoxyadenosine triphosphate
dTTP: Deoxythymidine triphosphate
dGTP: Deoxyguanosine triphosphate
dCTP: Deoxycytidine triphosphate
ATP: Adenosine triphosphate
EDTA: Ethylenediamine tetraacetic acid
SDS: Sodium dodecylphosphate
Gly: Glycine
Ala: Alanine
Val: Valine
Leu: Leucine
Ile: Isoleucine
Ser: Serine
Thr: Threonine
Cys: Cysteine
Met: Methionine
Glu: Glutamic acid
Asp: Aspartic acid
Lys: Lysine
Arg: Arginine
His: Histidine
Phe: Phenylalanine
Tyr: Tyrosine
Trp: Tryptophan
Pro: Proline
Asn: Asparagine
Gin: Glutamine
pGlu: Pyroglutaminic acid
*: Corresponding to termination codon
Me: Methyl group
Et: Ethyl group
Bu: Butyl group
Ph: Phenyl group
TC: Thiazolidine-4(R)-carboxamide group

Substituents, protective groups and reagents employed frequently in the specification are represented by the following codes.
Tos: p-Toluenesulfonyl
CHO: Formyl
Bzl: Benzyl
C₂Bzl: 2,6-Dichlorobenzyl
Bom: Benzyloxymethyl
Z: Benzyloxycarbonyl
Cl-Z: 2-Chlorobenzyloxycarbonyl
Br-Z: 2-Bromobenzyloxycarbonyl
Boc: t-Butoxycarbonyl
DNP: Dinitrophenyl
Trt: Trityl
Bum: t-Butoxymethyl
Fmoc: N-9-Fluorenylmethoxycarbonyl
HOBt: 1-Hydroxybenztriazole
HOOBt: 3,4-Dihydro-3-hydroxy-4-oxo-1,2,3-benzotriazine
HONB: 1-Hydroxy-5-norbornene-2,3-dicarboxyimide
DCC: N,N′-Dicyclohexylcarbodiimide

The SEQ. ID. No. in the sequence listing in this specification represent the following sequences.

SEQ ID NO.1 represents the amino acid sequence of a human novel G protein-conjugating receptor protein TGR6 of the invention.

SEQ ID NO.2 represents the nucleotide sequence of a cDNA encoding a human novel G protein-conjugating receptor protein TGR6 of the invention.

SEQ ID NO.3 represents the nucleotide sequence of Primer 1 employed in the PCR reaction in Example 1 described below.

SEQ ID NO.4 represents the nucleotide sequence of Primer 2 employed in the PCR reaction in Example 1 described below.

SEQ ID NO.5 represents the nucleotide sequence of the forward primer employed in the TaqMan PCR reaction in Example 2 described below.

SEQ ID NO.6 represents the nucleotide sequence of the reverse primer employed in the TaqMan PCR reaction in Example 2 described below.

SEQ ID NO.7 represents the nucleotide sequence of the probe employed in the TaqMan PCR reaction in Example 2 described below.

A transformant Escherichia coli TOP10/pCR2.1-TGR6 obtained in Example 1 described below has been deposited under Budapest treaty to International Patent Organism Depositary of National Institute of Advanced Industrial Science and Technology (IPOD) (Higashi 1-1-1, Tsukuba, Ibaraki) since May 1, 2000 under the deposition No.FERM BP-7152, and also to Institute for Fermentation (IFO) (2-17-85, JUSOHONMACHI, YODOGAWA-Ku, Osaka) since Apr. 20, 2000 under the deposition No.IFO 16425.

The present invention is further described in detail in the following Examples, which are not intended to restrict the invention. A gene engineering procedure employing E.coli was in accordance with the methods described in Molecular cloning.

### EXAMPLE 1

Cloning and Nucleotide Sequence Determination of cDNA Encoding Human brain-derived G Protein-conjugating Receptor Protein

A PCR was performed using a human brain cDNA (CLONTECH) as a template together with two primers, namely, Primer 1 (SEQ ID NO.3) and Primer 2 (SEQ ID NO.4). The reaction mixture in this PCR contained a 3 μl of the cDNA described above as a template, 1 μl of Advantage-GC2 polymerase Mix (CLONTECH), each 0.2 μM of Primer 1 (SEQ ID NO.3) and Primer 2 (SEQ ID NO.4), 200 ∞M of dNTPmix, 10 μl of the buffer attached to the enzyme and 5 μl of GC Melt, in the total volume of 50 μL. The PCR reaction involved 1 minute at 95° C., 5 cycles of 30 seconds at 95° C. followed by 2 minutes at 68° C., 5 cycles of 30 seconds at 95° C. followed by 30 seconds at 66° C. followed by 2 minutes at 68° C., 30 cycles of 30 seconds at 95° C. followed by 30 seconds at 64° C. followed by 2 minutes at 68° C., and a final chain elongation for 7 minutes at 68° C. After this PCR, the reaction product was cloned to a plasmid vector pCR2.1 (INVITROGEN) according to the instruction attached to a TOPO-TA cloning kit (INVITROGEN). This was then transduced into E.coli TOP10, and a clone having a cDNA was screened for in an LB agar medium containing ampicillin. Each clone was sequenced to obtain a cDNA (SEQ ID NO.2) encoding a novel G protein-conjugating receptor protein. A novel G protein-conjugating receptor protein containing the amino acid sequence derived from the nucleotide sequence represented by SEQ ID NO.2 was designated as TGR6. A transformant transformed with a DNA containing a DNA having the nucleotide sequence represented by SEQ ID NO.2 was designated as E.coli (Escherichia coli) TOP10/pCR2.1-TGR6.

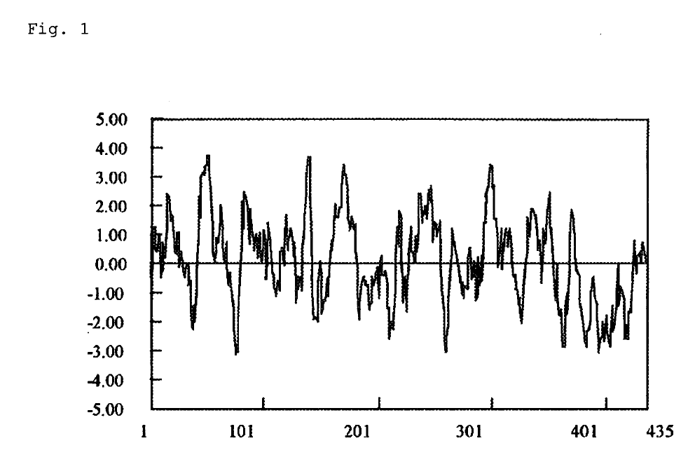

The hydrophobicity plotting of TGR6 is shown in FIG. 1.

### EXAMPLE 2

Analysis of Tissue Distribution of TGR6 Expression Using TaqMan PCR

Primers and probe were designed using Primer Express ver.1.0 (PE BIOSYSTEMS JAPAN), whereby producing a forward primer TGR6TQF (5′-TTAGC CCCTC AATTC TGCCG-3′, (SEQ ID NO.5)), a reverse primer TGR6TQR (5′-AGGTT GAGAC CCCAG CCCA-3′, (SEQ ID NO.6)) and a probe TGR6TQP (5′-CGGCG CCCAA CATTT CCGTG-3′, (SEQ ID NO.7)). As a reporter dye of the probe, FAM (6-carboxyfluorescein) was added.

For a standard cDNA, pCR2.1-TGR6 was used as a template together with Primer 1 (SEQ ID NO.3) and Primer 2 (SEQ ID NO.4) to effect amplification to obtain a PCR fragment, which was then purified using QIAquick PCR Purification Kit [QIAGEN (Germany)], and then adjusted at 10⁰ to 10⁶ copies/5 μl.

For cDNA sources of relevant tissues, Human Tissue cDNA Panel I and Panel II [CLONTECH Laboratories, Inc. (CA, USA)] were employed.

TaqMan PCR employed the reagents of TaqMan Universal PCR Master Mix (PE BIOSYSTEMS JAPAN) and reacted in ABI PRISM 7700 Sequence Detection System (PE BIOSYSTEMS JAPAN) according to the attached instructions.

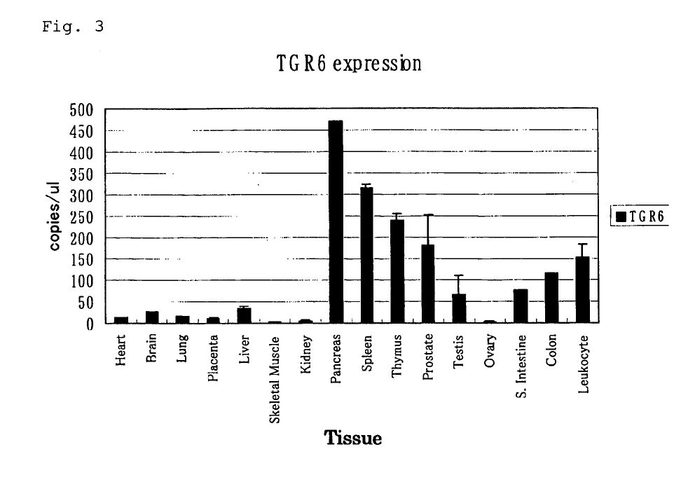

The results are shown in FIG. 3 and Table 1. THR 6 was expressed highly in pancreas and spleen.

TABLE 1

| Tissue | Expression (copies/μl) |
|---|---|
| Heart | 13 |
| Brain | 25 |
| Lung | 15 |
| Placenta | 11 |
| Liver | 34 |
| Skeletal muscle | 3 |
| Kidney | 6 |
| Pancreas | 470 |
| Spleen | 315 |
| Thymus | 240 |
| Prostate | 182 |
| Testis | 65 |
| Ovary | 3 |
| Small intestine | 76 |
| Large intestine | 116 |
| Leukocyte | 152 |

Industrial Applicability

A G protein-conjugating receptor protein of the present invention or a partial peptide and a salt thereof, and a polynucleotide encoding such the receptor protein or the partial peptide thereof (for example, DNA, RNA or its derivative) is useful in [1] determination of a ligand (agonist), [2] obtaining an antibody and an antiserum, [3] construction of a recombinant receptor protein expression system, [4] development of a receptor binding assay system and screening for a pharmaceutical candidate compound using this expression system, [5] designing a drug based on the comparison with a structurally-related ligand/receptor, [6] a reagent for preparing a probe or a PCR primer in a gene diagnosis, [7] creating a transgenic animal, or [8] a pharmaceutical such as a genetically prophylactic or therapeutic agent.

Sequence Listing Free Text

SEQ ID NO: 3
Designed oligonucleotide primer to amplify DNA encoding TGR6

SEQ ID NO: 4
Designed oligonucleotide primer to amplify DNA encoding TGR6

SEQ ID NO: 5
Designed oligonucleotide forward primer for TaqMan PCR

SEQ ID NO: 6
Designed oligonucleotide reverse primer for TaqMan PCR

SEQ ID NO: 7
Designed oligonucleotide probe for TaqMan PCR

1. An isolated g protein-conjugating receptor protein comprising an amino acid sequence identical or substantially identical to the amino acid sequence represented by SEQ ID NO.1, or a salt thereof:

2. A partial peptide of the G protein-conjugating receptor protein according to claim 1, or a salt thereof.

3. An isolated polynucleotide comprising a polynucleotide encoding the G protein-conjugating receptor protein according to claim 1.

4. The polynucleotide according to claim 3 which is a DNA.

5. The polynucleotide according to claim 3 having the nucleotide sequence represented by SEQ ID NO.2.

6. A recombinant vector comprising the polynucleotide according to claim 3.

7. A transformant transformed with the recombinant vector according to claim 6.

8. A method for producing the G protein-conjugating receptor protein according to claim 1 or a salt thereof, comprising cultivating the transformant having an expressible recombinant vector comprising a nucleic acid which encodes for said protein, under suitable conditions for producing said protein, and for sufficient time to generate the G protein-conjugating receptor protein according to claim 1.

9. An antibody against the G protein-conjugating receptor protein according to claim 1 or the partial peptide or a salt thereof.

10. The antibody according to claim 9 which is a neutralizing antibody which inactivates the signal transmission of the G protein-conjugating receptor.

11. A diagnostic composition comprising the antibody according to claim 9.

12. A ligand for the G protein-conjugating receptor protein according to claim 1 or a salt thereof which is obtainable by using the G protein-conjugating receptor protein according to claim 1 or the partial peptide or a salt thereof in a screening assay.

13. A pharmaceutical composition comprising a ligand for the G protein-conjugating receptor protein according to claim 12 and a pharmaceutically acceptable carrier, diluent or excipient.

14. A method for determining a ligand for the G protein-conjugating receptor protein according to claim 1 or a salt thereof, comprising using the G protein-conjugating receptor protein according to claim 1 or the partial peptide or a salt thereof in a screening assay.

15. A method for screening a compound or a salt thereof which is capable of altering the binding affinity between a ligand and the G protein-conjugating receptor protein according to claim 1 or a salt thereof, comprising contacting said compound with said ligand or said protein or the partial or a salt thereof, and detecting any alteration in binding between said ligand and said protein.

16. A screening kit for a compound or a salt thereof capable of altering the binding affinity between a ligand and the G protein-conjugating receptor protein according to claim 1 or a salt thereof, comprising the G protein-conjugating receptor protein according to claim 1 or the partial peptide or a salt thereof.

17. A compound or a salt thereof capable of altering the binding affinity between a ligand and the G protein-conjugating receptor protein comprising an amino acid sequence identical or substantially identical to the amino acid sequence represented by SEQ ID NO.1 or a salt thereof, which is obtainable by using the screening method according to claim 15.

18. A pharmaceutical composition comprising a compound or a salt thereof of claim 17 and a pharmaceutically acceptable carrier, diluent or excipient.

19. An isolated polynucleotide hybridizing with the polynucleotide according to claim 3 under a high stringent condition.

20. An isolated polynucleotide comprising a nucleotide sequence or a part thereof which is complementary with the polynucleotide according to claim 3.

21. A method for quantifying an mRNA of the G protein-conjugating receptor protein according to claim 1, comprising using the polynucloeotide or a part thereof encoding for said protein as a probe.

22. A method for quantifying the G protein-conjugating receptor protein according to claim 1, comprising using an antibody which binds to said protein in an assay.

23. A method for diagnosing a disease related to a function of the G protein-conjugating receptor protein according to claim 1, comprising using a quantification method and detecting said protein or mRNA encoding for said protein.

24. A screening method for a compound or a salt thereof capable of altering the expression level of the G protein-conjugating receptor protein according to claim 1, comprising combining said compound with a transformed host cell which can express said protein, and detecting mRNA encoding for said protein.

25. A screening method for a compound or a salt thereof capable of altering the amount of the G protein-conjugating receptor protein according to claim 1 in a cell membrane, comprising combining said compound with a transformed host cell which can express said protein, and detecting said protein.

26. A compound or a salt thereof capable of altering the expression level of the G protein-conjugating receptor protein comprising an amino acid sequence identical or substantially identical to the amino acid sequence represented by SEQ ID NO.1, which is obtainable by using the screening method according to claim 24.

27. A compound or a salt thereof capable of altering the amount of the G protein-conjugating receptor protein comprising an amino acid sequence identical or substantially identical to the amino acid sequence represented by SEQ ID NO.1 in a cell membrane, which is obtainable by using the screening method according to claim 25.

28. A pharmaceutical composition comprising a compound or a salt thereof capable of altering the expression level of the G protein-conjugating receptor protein comprising an amino acid sequence identical or substantially identical to the amino acid sequence represented by SEQ ID NO.1, which is obtainable by using the screening method according to claim 24.

29. A pharmaceutical composition comprising a compound or a salt thereof capable of altering the amount of the G protein-conjugating receptor protein comprising an amino acid sequence identical or substantially identical to the amino acid sequence represented by SEQ ID NO.1 in a cell membrane, which is obtainable by using the screening method according to claim 25.

30. A pharmaceutical composition for preventing or treating a disease in central nervous system, inflammatory disease, circulatory disease, cancer, diabetes or infertility comprising an effective amount of an inhibitor of a G protein-conjugating receptor protein comprising an amino acid sequence identical or substantially identical to the amino acid sequence represented by SEQ ID NO.1, and a pharmaceutically acceptable carrier, diluent or excipient.

31. A method for preventing or treating a disease in the central nervous system, inflammatory disease, circulatory disease, cancer, diabetes or infertility, comprising administering to a mammal in need thereof an effective amount of a compound or a salt thereof capable of altering the binding affinity between a ligand and the G protein-conjugating receptor protein comprising an amino acid sequence identical or substantially identical to the amino acid sequence represented by SEQ ID NO.1 or a salt thereof, which is obtainable by using the screening method according to claim 15.

32. A method for preventing or treating a disease in the central nervous system, inflammatory disease, circulatory disease, cancer, diabetes or infertility, comprising administering to a mammal in need thereof an effective amount of a compound or a salt thereof capable of altering the expression level of the G protein-conjugating receptor protein comprising an amino acid sequence identical or substantially identical to the amino acid sequence represented by SEQ ID NO.1, which is obtainable by using the screening method according to claim 24.

33. A method for preventing or treating a disease in the central nervous system, inflammatory disease, circulatory disease, cancer, diabetes or infertility, comprising administering to a mammal in need thereof an effective amount of a compound or a salt thereof capable of altering the amount of the G protein-conjugating receptor protein comprising an amino acid sequence identical or substantially identical to the amino acid sequence represented by SEQ ID NO.1 in a cell membrane, which is obtainable by using the screening method according to claim 25.

34. A method for making a pharmaceutical for preventing or treating a disease in central nervous system, inflammatory disease, circulatory disease, cancer, diabetes or infertility comprising combining a compound or a salt thereof capable of altering the binding affinity between a ligand and the G protein-conjugating receptor protein comprising an amino acid sequence identical or substantially identical to the amino acid sequence represented by SEQ ID NO.1 or a salt thereof, which is obtainable by using the screening method according to the claim 15, with a pharmaceutically acceptable carrier, diluent or excipient.

35. A method for making a pharmaceutical for preventing or treating a disease in central nervous system, inflammatory disease, circulatory disease, cancer, diabetes or infertility comprising combining a compound or a salt thereof capable of altering the binding affinity between a ligand and the G protein-conjugating receptor protein comprising an amino acid sequence identical or substantially identical to the amino acid sequence represented by SEQ ID NO.1 or a salt thereof, which is obtainable by using the screening method according to the claim 24, with a pharmaceutically acceptable carrier, diluent or excipient.

36. A method for making a pharmaceutical for preventing or treating a disease in central nervous system, inflammatory disease, circulatory disease, cancer, diabetes or infertility comprising combining a compound or a salt thereof capable of altering the binding affinity between a ligand and the G protein-conjugating receptor protein comprising an amino acid sequence identical or substantially identical to the amino acid sequence represented by SEQ ID NO.1 or a salt thereof, which is obtainable by using the screening method according to the claim 25, with a pharmaceutically acceptable carrier, diluent or excipient.


SEQUENCE LISTING (7 sequences)

SEQ ID NO: 1 - protein, 434 aa, Human [sequence omitted]

SEQ ID NO: 2 - DNA, 1302 bp, Human
atggaggatctctttagcccctcaattctgccgccggcgcccaacatttccgtgcccatcttgctgggctggggtctcaacctgaccttggggcaaggagcccctgcctctgggccgcccagccgccgcgtccgcctggtgttcctgggggtcatcctggtggtggcggtggcaggcaacaccacagtgctgtgccgcctgtgcggcggcggcgggccctgggcgggccccaagcgtcgcaagatggacttcctgctggtgcagctggccctggcggacctgtacgcgtgcgggggcacggcgctgtcacagctggcctgggaactgctgggcgagccccgcgcggccacgggggacctggcgtgccgcttcctgcagctgctgcaggcatccgggcggggcgcctcggcccacctcgtggtgctcatcgccctcgagcgccggcgcgcggtgcgtcttccgcacggccggccgctgcccgcgcgtgccctcgccgccctgggctggctgctggcactgctgctggcgctgcccccggccttcgtggtgcgcggggactccccctcgccgctgccgccgccgccgccgccaacgtccctgcagccaggcgcgcccccggccgcccgcgcctggccggggcagcgtcgctgccacgggatcttcgcgcccctgccgcgctggcacctgcaggtctacgcgttctacgaggccgtcgcgggcttcgtcgcgcctgttacggtcctgggcgtcgcttgcggccacctactctccgtctggtggcggcaccggccgcaggcccccgcggctgcagcgccctggtcggcgagcccaggtcgagcccctgcgcccagcgcgctgccccgcgccaaggtgcagagcctgaagatgagcctgctgctggcgctgctgttcgtgggctgcgagctgccctactttgccgcccggctggcggccgcgtggtcgtccgggcccgcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttc

SEQ ID NO: 3 - DNA, 24 bp, Artificial Sequence
  Designed oligonucleotide primer to amplify DNA encoding TGR6
cgcaaagccgcccgaatccatctc

SEQ ID NO: 4 - DNA, 24 bp, Artificial Sequence
  Designed oligonucleotide primer to amplify DNA encoding TGR6
aggagagtggaggaaggggacaac

SEQ ID NO: 5 - DNA, 20 bp, Artificial Sequence
  Designed oligonucleotide forward primer for TaqMan PCR
ttagcccctcaattctgccg

SEQ ID NO: 6 - DNA, 19 bp, Artificial Sequence
  Designed oligonucleotide reverse primer for TaqMan PCR
aggttgagaccccagccca

SEQ ID NO: 7 - DNA, 20 bp, Artificial Sequence
  Designed oligonucleotide probe for TaqMan PCR
cggcgcccaacatttccgtg